(19) United States
(12) Patent Application Publication
(10) Pub. No.: US 2024/0391958 A1
(43) Pub. Date: Nov. 28, 2024

(54) KEANUMYCINS AND USES THEREOF IN MEDICINE AND PLANT PROTECTION

(71) Applicant: Leibniz-Institut für Naturstoff-Forschung und Infektionsbiologie e. V. Hans-Knöll-Institut (HKI), JENA (DE)

(72) Inventors: Sebastian PFLANZE, JENA (DE); Sebastian GÖTZE, JENA (DE); Pierre STALLFORTH, LEIPZIG (DE)

(21) Appl. No.: 18/672,777

(22) Filed: May 23, 2024

Related U.S. Application Data
(60) Provisional application No. 63/468,871, filed on May 25, 2023.

(51) Int. Cl. C07K 7/54; A61K 38/00; A61P 31/04; A61P 33/04; C07K 1/20; C12P 21/02; C12R 1/38

(52) U.S. Cl. CPC C07K 7/54; A61P 31/04; A61P 33/04; C07K 1/20; C12P 21/02; A61K 38/00; C12R 2001/38

(57) ABSTRACT

The present invention relates to a new class of antibiotic substances, designated as keanumycins for their versatility in fighting and controlling fungi and amoebas in mammals and plants.

### CROSS REFERENCE TO RELATED APPLICATIONS

The present application claims the benefit of U.S. Provisional Application Ser. No. 63/468,871, filed May 25, 2023, which is hereby incorporated by reference herein in its entirety, including any figures, tables, or drawings.

### FIELD OF THE INVENTION

The present invention relates to a new class of antibiotic substances, designated as keanumycins for their versatility in fighting and controlling fungi and amoebas in mammals and plants.

### BACKGROUND OF THE INVENTION

Over- and misuse of anti-infectives in health care, livestock, and plant agriculture has led to the increase and global distribution of antimicrobial resistance in microorganisms. The worrisome prevalence of multidrug-resistant pathogens is the cause of the antimicrobial resistance crisis, which has severe socioeconomic implications for human society. In the United States alone, approximately 3 million individuals are infected with antimicrobial-resistant germs each year, causing 35,000 causalities. In addition, many patients will suffer from severely debilitating and often long-term side effects from the protracted illness and intensive treatment. Combating antimicrobial resistance relies on a combination of strategies ranging from the reduction of anti-infective use in general, as well as creating new antimicrobials. Importantly, treatment options for stockbreeding and plant agriculture that do not rely on antimicrobials used in human medicine are desperately needed.

Unfortunately, the development of antimicrobials has been discontinued by a majority of pharmaceutical companies as it was deemed unprofitable. One of the main reasons for this decline can be attributed to the fact that the majority of all marketed antibiotics are natural products or their derivatives. Unfortunately, the costs associated with finding new natural product-based lead structures have increased significantly, due to extraordinary rediscovery rates of already known substances or compound classes.

In recent times, Lipopeptides (LPs) have gained a lot of attention from the science community respective of their vast applications. Lipopeptides have turned out to be one of the most important secondary metabolites produced by microorganisms leading to growing research interest in them. With more than 200 different LPs identified to date, they are structurally diverse compounds. The high structural variability is the resultant of frequently occurring amino acid substitutions. This characteristic feature of LPs in turn gives them the ability to decrease interfacial and surface tension. Structurally, they are low molecular weight compounds that consist of a fatty acid acyl chain (hydrophobic) attached to a peptide head (hydrophilic) (Mukherjee, D., Rooj, B., Mandal, U. (2021). Antibacterial Biosurfactants. In: Inamuddin, Ahamed, M. I., Prasad, R. (eds) Microbial Biosurfactants. Environmental and Microbial Biotechnology. Springer, Singapore. https://doi.org/10.1007/978-981-15-6607-3_13). The fatty acid chain does not exceed more than 17 carbons in length, whereas the number of amino acids ranges anywhere between 7 and 35. Most documented LPs are produced from Pseudomonas-(Proteobacteria) and Bacillus-(firmicutes) strains. Other strains reported to produce LPs are Streptomyces and some fungal strains.

Vestola J. et al. (in: Hassallidins, antifungal glycolipopeptides, are widespread among cyanobacteria and are the end-product of a nonribosomal pathway. Proc Natl Acad Sci USA. 2014 May 6; 111 (18): E1909-17. doi: 10.1073/pnas.1320913111. Epub 2014 Apr. 17. PMID: 24742428; PMCID: PMC4020101) disclose that cyanobacteria produce a wide variety of cyclic peptides, including the widespread hepatotoxins microcystins and nodularins. Another class of peptides, cyclic glycosylated lipopeptides called hassallidins, show antifungal activity. Previously, two hassallidins (A and B) were reported from an epilithic cyanobacterium Hassallia sp. and found to be active against opportunistic human pathogenic fungi. Bioinformatic analysis of the Anabaena sp. 90 genome identified a 59-kb cryptic inactive nonribosomal peptide synthetase gene cluster proposed to be responsible for hassallidin biosynthesis. They describe the hassallidin biosynthetic pathway from Anabaena sp. SYKE748A, as well as the large chemical variation and common occurrence of hassallidins in filamentous cyanobacteria. Analysis demonstrated that 20 strains of the genus Anabaena carry hassallidin synthetase genes and produce a multitude of hassallidin variants that exhibit activity against Candida albicans. The compounds discovered here were distinct from previously reported hassallidins A and B. The IC50 of hassallidin D was 0.29-1.0 μM against Candida strains. A large variation in amino acids, sugars, their degree of acetylation, and fatty acid side chain length was detected. In addition, hassallidins were detected in other cyanobacteria including Aphanizomenon, Cylindrospermopsis raciborskii, Nostoc, and Tolypothrix. These compounds may protect some of the most important bloom-forming and globally distributed cyanobacteria against attacks by parasitic fungi.

Mareš J, et al. (in: Alternative Biosynthetic Starter Units Enhance the Structural Diversity of Cyanobacterial Lipopeptides. Appl Environ Microbiol. 2019 Feb. 6; 85 (4): c02675-18. doi: 10.1128/AEM.02675-18. PMID: 30504214; PMCID: PMC6365827) disclose puwainaphycins (PUWs) and minutissamides (MINs) as structurally analogous cyclic lipopeptides possessing cytotoxic activity. Both types of compound exhibit high structural variability, particularly in the fatty acid (FA) moiety. Although a biosynthetic gene cluster responsible for synthesis of several PUW variants has been proposed in a cyanobacterial strain, the genetic background for MINs remains unexplored. They report PUW/MIN biosynthetic gene clusters and structural variants from six cyanobacterial strains. They deciphered an important biosynthetic trait of a prominent group of bioactive lipopeptides and revealed evidence for initiation of biosynthesis by two alternative starter units hardwired directly in the same gene cluster, eventually resulting in the production of a remarkable range of lipopeptide variants. They identified several unusual tailoring genes potentially involved in modifying the fatty acid chain. Careful characterization of these biosynthetic gene clusters and their diverse products could provide important insight into lipopeptide biosynthesis in prokaryotes. Some of the variants identified exhibit cytotoxic and antifungal properties, and some are associated with a toxigenic biofilm-forming strain.

Michelsen C F, et al. (in: Nonribosomal peptides, key biocontrol components for Pseudomonas fluorescens In5, isolated from a Greenlandic suppressive soil. mBio. 2015 Mar. 17; 6 (2): e00079. doi: 10.1128/mBio.00079-15. PMID: 25784695; PMCID: PMC4453515) document that a potato soil at Inneruulalik in southern Greenland is suppressive against Rhizoctonia solani Ag3 and uncover the suppressive antifungal mechanism of a highly potent biocontrol bacterium, Pseudomonas fluorescens In5, isolated from the suppressive potato soil. A combination of molecular genetics, genomics, and matrix-assisted laser desorption ionization-time of flight (MALDI-TOF) imaging mass spectrometry (IMS) revealed an antifungal genomic island in P. fluorescens In5 encoding two nonribosomal peptides, nunamycin and nunapeptin, which are key components for the biocontrol activity by strain In5 in vitro and in soil microcosm experiments. Further characterization of the two peptides revealed nunamycin to be a monochlorinated 9-amino-acid cyclic lipopeptide with similarity to members of the syringomycin group, whereas nunapeptin was a 22-amino-acid cyclic lipopeptide with similarity to corpeptin and syringopeptin.

In the context of the increasing resistance of pathogenic organisms against antibiotic substances, urgently new active moieties and compounds are needed. It is therefore an object of the present invention to provide a new class of antibiotic substances, their uses and methods for producing them. Other objects and advantages will readily become apparent for the person of skill from studying the following more detailed description and examples.

### BRIEF SUMMARY

The object of the present invention is solved by providing a lipopeptide according to the following general formula 1,

(Formula 1)
X-L-Ser-D-Dab-Gly-D-Hse-L-Dab-L-allo-Thr-Dhb-L-
Asp-L-Thr

wherein Dhb stands for dehydrobutyrine.
X is selected from CH₃—(CH₂)₁₄—CONH, CH₃—(CH₂)₁₂—CHOH—CH₂—CONH, and CH₃—(CH₂)₁₁—CHOH—CHOH—CH₂—CONH,
and
wherein the molecule optionally forms a cycle between the L-Ser and the last L-Thr moiety, and pharmaceutically or agrochemically acceptable salts thereof.

Preferred is the lipopeptide according to the present invention, wherein at least one of the L-Thr is replaced by 4-Cl-L-Thr, and/or wherein the L-Asp is replaced by L-β-threo-OH-Asp. Further preferred is the lipopeptide according to the present invention. selected from the group consisting of

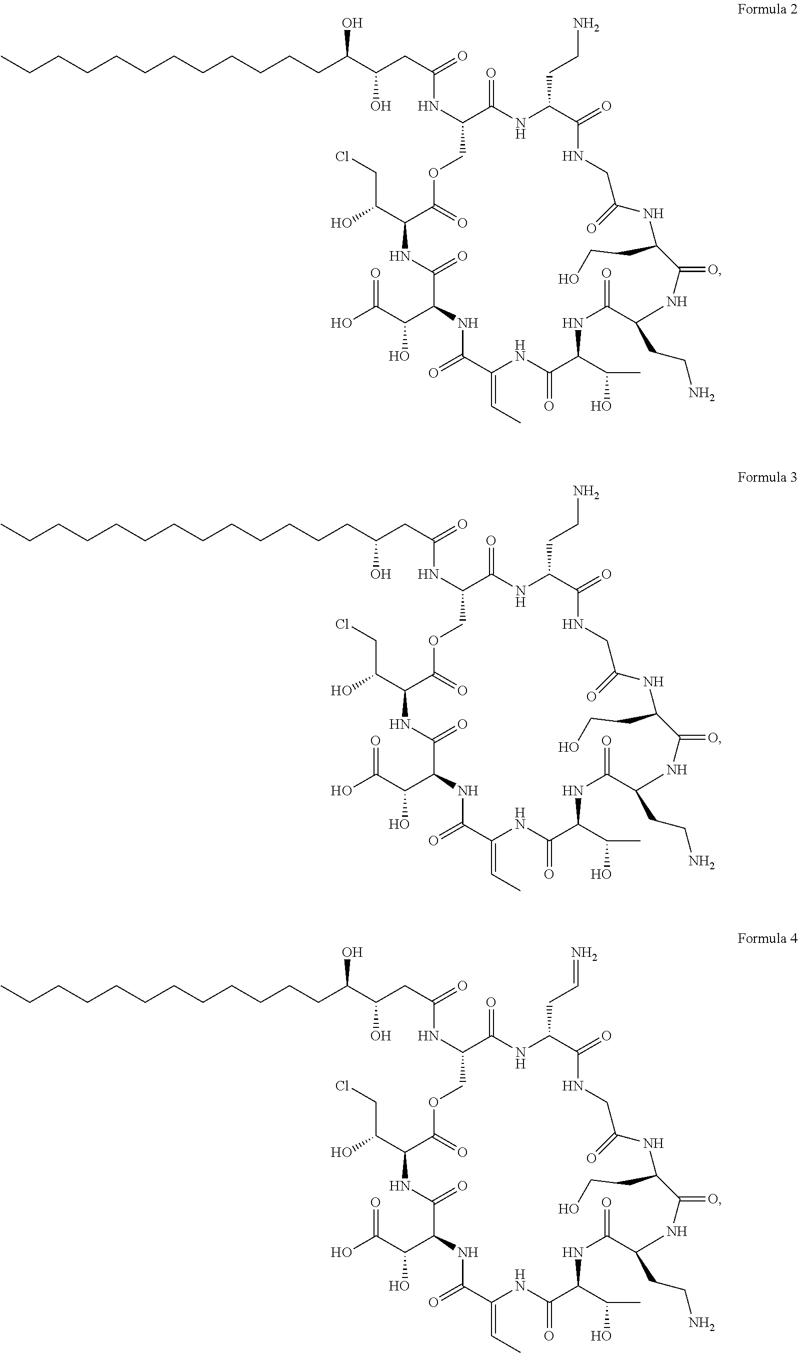

and pharmaceutically or agrochemically acceptable salts thereof.

According to another aspect of the present invention, the above object is solved by a method for producing the lipopeptide according to the present invention, comprising a) suitably culturing a suitable strain of the bacterium Pseudomonas, such as, for example, Pseudomonas sp. QS1027, b) extracting the supernatant of the culture with an organic solvent, such as, for example, n-butanol, c) dissolving the dried extract of b) in alcohol, such as, for example, MeOH, d) fractioning the extract of c) using reverse phase chromatography and elution with 75% (v/v) acetonitrile in water containing 0.1% (v/v) formic acid, and e) final purification using preparative HPLC.

According to another aspect of the present invention, the above object is solved by a method for producing the lipopeptide according to the present invention, comprising chemical synthesis of the fatty acid chain and the amino acid chain comprising use of the synthetic amino acid building block 2-amino-4-iminobutanoic acid.

In yet another aspect of the present invention, the above object is solved by a pharmaceutical composition or a plant protective composition comprising at least one lipopeptide according to the present invention or as produced according to the present invention, together with at least one pharmaceutically or agrochemically acceptable carrier or diluent, or at least one plant protective composition acceptable carrier or diluent.

According to another aspect of the present invention, the above object is solved by an antimycotic or antiamoebal or amoebicidal composition comprising at least one lipopeptide according to the present invention comprising a supernatant of a culture of a suitable strain of the bacterium Pseudomonas, such as, for example, Pseudomonas sp. QS1027, in particular a sterile filtrate of the supernatant.

According to another aspect of the present invention, the above object is solved by the pharmaceutical composition or a plant protective composition according to the present invention or the antimycotic composition according to the present invention for use as a plant protective composition, for example in a crop plant, such as soy bean.

According to another aspect of the present invention, the above object is solved by a method for preventing or inhibiting the growth of a fungus or an amoeba, comprising contacting the fungus or the amoeba with the pharmaceutical composition or the plant protective composition according to the present invention or the antimycotic or antiamoebal composition according to the present invention. Preferred is the method according to the present invention, wherein said fungus is a plant pathogen, such as, for example, B. cinerea, Phakopsora pachyrhizi or Alternaria solani, and wherein preferably said method comprises the step of applying said pharmaceutical composition or the plant protective composition according to the present invention or the antimycotic composition according to the present invention onto a plant.

According to another aspect of the present invention, the above object is solved by a method for preventing or inhibiting the growth of a fungus or an amoeba, wherein the concentration of the lipopeptide in the pharmaceutical composition or the plant protective composition according to the present invention or the antimycotic or antiamoebal composition according to the present invention is in a range of about 0.05 μM to about 1.5 M, preferably about 0.05 μM to about 1 μM, and most preferably at about 0.1 μM.

According to another aspect of the present invention, the above object is solved by a method for preventing or treating a fungal or an amoebal infection in a subject in need of said prevention or treatment, comprising administering to said subject an effective amount of the at least one lipopeptide according to the present invention or as produced according to the present invention or the pharmaceutical composition according to the present invention. Preferred is the method according to the present invention, wherein the fungal infection is caused by Candida spp., in particular Candida albicans or Candida auris, Sporobolomyces salmonicolor, or Penicillium notatum or wherein the amoebal infection is caused by Dictyostelium discoideum, Acanthamoeba castellanii, or Acanthamoeba comandoni.

### DETAILED DESCRIPTION

As mentioned above, in a first aspect of the present invention, the object of the present invention is solved by a lipopeptide according to the following general formula 1,

(Formula 1)
X-L-Ser-D-Dab-Gly-D-Hse-L-Dab-L-allo-Thr-Dhb-L-
Asp-L-Thr

wherein
X is selected from CH₃—(CH₂)₁₄—CONH, CH₃—(CH₂)₁₂—CHOH—CH₂—CONH, and CH₃—(CH₂)₁₁—CHOH—CHOH—CH₂—CONH,
and
wherein the molecule optionally forms a cycle between the L-Ser and the last L-Thr moiety, and pharmaceutically or agrochemically acceptable salts thereof.

Götze S, et al. (in: Ecological Niche-Inspired Genome Mining Leads to the Discovery of Crop-Protecting Nonribosomal Lipopeptides Featuring a Transient Amino Acid Building Block. J Am Chem Soc. 2023 Feb. 1; 145 (4): 2342-2353. doi: 10.1021/jacs.2c11107. Epub 2023 Jan. 20. PMID: 36669196; PMCID: PMC9897216, which is incorporated by reference in its entirety) discloses that investigating the ecological context of microbial predator-prey interactions enables the identification of microorganisms, which produce multiple secondary metabolites to evade predation or to kill the predator. In addition, genome mining combined with molecular biology methods can be used to identify further biosynthetic gene clusters that yield new antimicrobials to fight the antimicrobial crisis. In contrast, classical screening-based approaches have limitations since they do not aim to unlock the entire biosynthetic potential of a given organism. They describe the genomics-based identification of keanumycins A-C. These nonribosomal peptides enable bacteria of the genus Pseudomonas to evade amoebal predation. While being amoebicidal at a nanomolar level, these compounds also exhibit a strong antimycotic activity in particular against the devastating plant pathogen Botrytis cinerea and they drastically inhibit the infection of Hydrangea macrophylla leaves using only supernatants of Pseudomonas cultures. The structures of the keanumycins were fully elucidated through a combination of nuclear magnetic resonance, tandem mass spectrometry, and degradation experiments revealing an unprecedented terminal imine motif in keanumycin C extending the family of nonribosomal amino acids by a highly reactive building block. In addition, chemical synthesis unveiled the absolute configuration of the unusual dihydroxylated fatty acid of keanumycin A, which has not yet been reported for this lipodepsipeptide class. Finally, a detailed genome-wide microarray analysis of Candida albicans exposed to keanumycin A shed light on the mode-of-action of this potential natural product lead, which will aid the development of new pharmaceutical and agrochemical antifungals.

In the context of the present invention, the term “keanumycin” shall interchangeably designate a lipopeptide of the new class of lipopeptides as exemplified by keanumycins A-C, falling under the general formula I as above, in particular keanumycin A.

Preferred is the lipopeptide according to the present invention, wherein at least one of the L-Thr is replaced by 4-Cl-L-Thr, and/or wherein the L-Asp is replaced by L-β-threo-OH-Asp. Further preferred is the lipopeptide according to the present invention, selected from the group consisting of

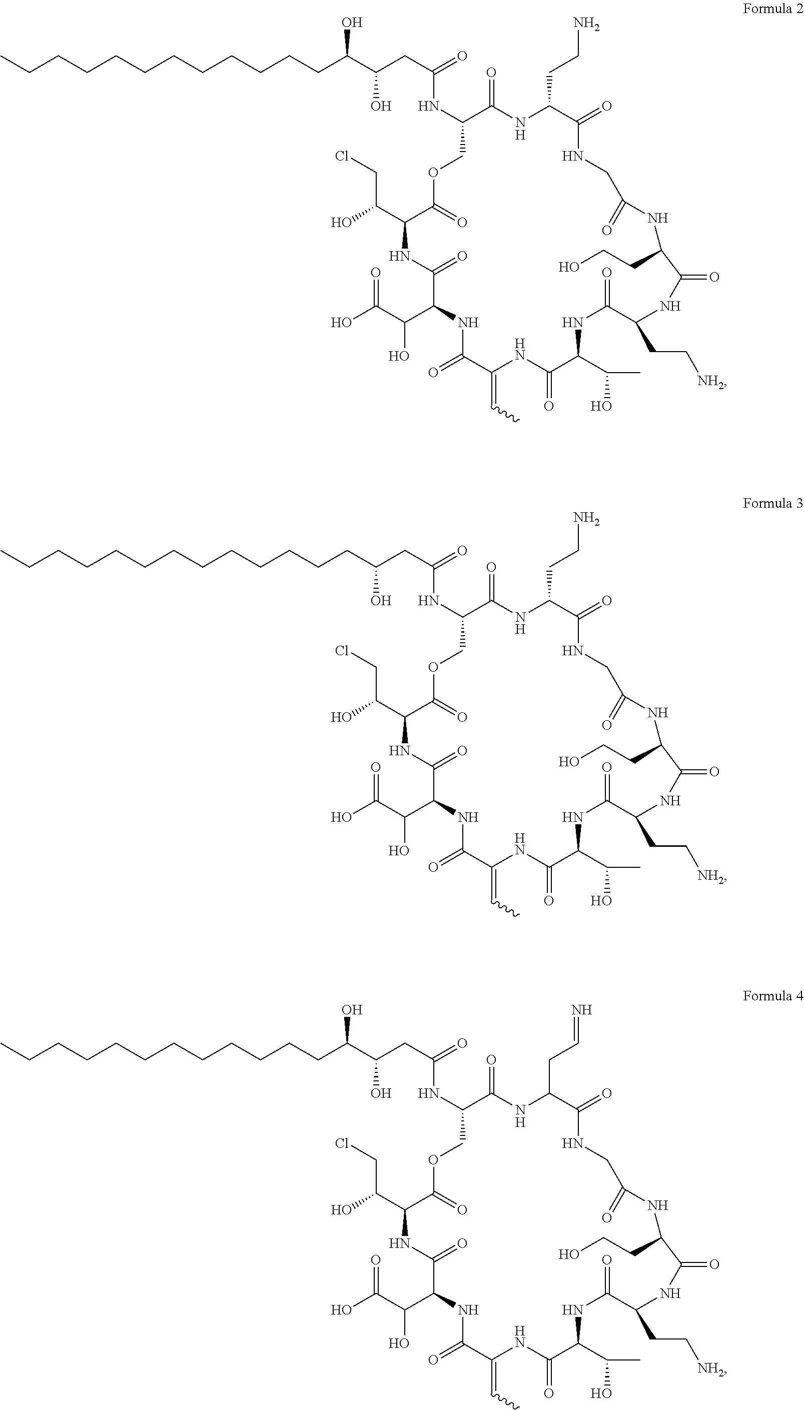

and pharmaceutically or agrochemically acceptable salts thereof.

Here, the inventors elucidated the structure of a new class of lipopeptides as exemplified by keanumycins A-C by using a combination of analytical as well as synthetic methods and discovered a novel amino acid building block, namely, 2-amino-4-iminobutanoic acid, which has not been described in an NRP. Keanumycin A is a strong amoebicide and antifungal that shows little cytotoxicity making it a good lead structure for the development of new antimycotics. Furthermore, the fermentation broth of the producing organism containing keanumycins can be directly applied to plants to stop the development of Botrytis blight or soybean rust (see FIG. 10) and has potential for the development of an ecofriendly alternative to antifungal agrochemicals. Studies of the mechanism of action revealed that keanumycin A most likely is a membrane-active compound that disrupts the fungal cell wall integrity leading to cell death.

Bacteria of the genus Pseudomonas produce a strong antimicrobial natural product. The substance is effective against both plant fungal diseases and human-pathogenic fungi. This newly discovered natural product group of keanumycins in bacteria works effectively against the plant pest Botrytis cinerea, which triggers gray mold rot and causes immense harvest losses every year, but the active ingredient also inhibits fungi that are dangerous to humans, such as Candida albicans. According to previous studies, it is harmless to plant and human cells.

Keanumycins are therefore an environmentally friendly alternative to chemical pesticides, and also offer an alternative in the fight against resistant fungi. Keanumycin is biodegradable, so no permanent residues should form in the soil. This means that the natural product has the potential to become an environmentally friendly alternative to chemical pesticides.

Keanumycin can also be used in humans. According to the tests conducted so far, the natural product is not highly toxic for human cells and is already effective against fungi in very low concentrations. This makes it a good candidate for the pharmaceutical development of new antimycotics. These are also urgently needed, as there are very few drugs against fungal infections on the market.

Chauhan V, et al. (in: Combination of classical and statistical approaches to enhance the fermentation conditions and increase the yield of Lipopeptide(s) by Pseudomonas sp. OXDC12: its partial purification and determining antifungal property. Turk J Biol. 2021 Dec. 14; 45 (6): 695-710. doi: 10.3906/biy-2106-59. PMID: 35068950; PMCID: PMC8733952) disclose that around 200 different lipopeptides (LPs) have been identified to date, most of which are produced via Bacillus and Pseudomonas species. The clinical nature of the lipopeptide (LP) has led to a big surge in its research. They show antimicrobial and antitumor activities due to which mass-scale production and purification of LPs are beneficial. Response surface methodology (RSM) approach has emerged as an alternative in the field of computational biology for optimizing the reaction parameters using statistical models. In the present study, Pseudomonas sp. strain OXDC12 was used for production and partial purification of LPs using Thin Layer Chromatography (TLC). The main goal of the study was to increase the overall yield of LPs by optimizing the different variables in the fermentation broth. This was achieved using a combination of one factor at a time (OFAT) and response surface methodology (RSM) approaches. OFAT technique was used to optimize the necessary parameters and was followed by the creation of statistical models (RSM) to optimize the remaining variables. Maximum mycelial growth inhibition (%) against the fungus Mucor sp. was 61.3% for LP. Overall, the combination of both OFAT and RSM helped in increasing the LPs yield by 3 folds from 367 mg/L to 1169 mg/L.

The lipopeptides according to the present invention can be produced using bacterial synthesis and subsequent purification. For this aspect of the present invention, first, a suitable strain of the bacterium Pseudomonas, such as, for example, Pseudomonas sp. QS1027 is grown in culture. Respective media and culture methods are known to the person of skill in the art.

Then, the supernatant of the culture is extracted with a suitable organic solvent, such as, for example, n-butanol. After drying, the dried extract of b) is then dissolved in alcohol, such as, for example, methanol, MeOH. Subsequently, the extract of c) is fractionated using reverse phase chromatography and an elution with 75% (v/v) acetonitrile in water containing 0.1% (v/v) formic acid. The fraction is then finally purified using preparative HPLC.

The lipopeptides according to the present invention can further be produced using chemical synthesis. According to an embodiment of the method for producing the lipopeptide according to the present invention, the method comprises the chemical synthesis of building blocks, such as the fatty acid chain and the amino acid chain comprising use of the synthetic amino acid building block 2-amino-4-iminobutanoic acid, with a subsequent combination of the blocks into the final lipopeptide, and ring formation. Exemplary pathways for such synthesis are disclosed in the state of the art and herein, and can be readily modified by the person of skill, if required.

Another aspect of the present invention then relates to a pharmaceutical composition or a plant protective composition comprising at least one lipopeptide according to the present invention or as produced according to the present invention, together with at least one pharmaceutically acceptable carrier or diluent, or at least one plant protective composition acceptable carrier or diluent.

The term “pharmaceutically or therapeutically acceptable excipient or carrier” refers to a solid or liquid filler, diluent or encapsulating substance which does not interfere with the effectiveness or the biological activity of the active ingredients and which is not substantially toxic to the host, which may be either humans or animals, to which it is administered. Depending upon the particular route of administration, a variety of pharmaceutically acceptable carriers such as those well known in the art may be used. Non-limiting examples include sugars, starches, cellulose and its derivatives, malt, gelatin, talc, calcium sulfate, vegetable oils, synthetic oils, polyols, alginic acid, phosphate buffered solutions, emulsifiers, isotonic saline, s and pyrogen-free water. Pharmaceutically acceptable carriers or excipients also include diluents (fillers, bulking agents, e.g. lactose, microcrystalline cellulose), disintegrants (e.g. sodium starch glycolate, croscarmellose sodium), binders (e.g. PVP, HPMC), lubricants (e.g. magnesium stearate), glidants (e.g. colloidal SiO₂), solvents/co-solvents (e.g. aqueous vehicle, Propylene glycol, glycerol), buffering agents (e.g. citrate, gluconates, lactates), preservatives (e.g. Na benzoate, parabens (Me, Pr and Bu), BKC), anti-oxidants (e.g. BHT, BHA, Ascorbic acid), wetting agents (e.g. polysorbates, sorbitan esters), thickening agents (e.g. methylcellulose or hydroxyethylcellulose), sweetening agents (e.g. sorbitol, saccharin, aspartame, acesulfame), flavoring agents (e.g. peppermint, lemon oils, butterscotch, etc.), humectants (e.g. propylene, glycol, glycerol, sorbitol). Other suitable pharmaceutically acceptable excipients are inter alia described in Remington's Pharmaceutical Sciences, 15^{th} Ed., Mack Publishing Co., New Jersey (1991) and Bauer et al., Pharmazeutische Technologic, 5^{th} Ed., Govi-Verlag Frankfurt (1997). The person skilled in the art knows suitable formulations for the compounds according to the present invention, and will readily be able to choose suitable pharmaceutically acceptable carriers or excipients, depending, e.g., on the formulation and administration route of the pharmaceutical composition.

All suitable modes of administration are contemplated according to the invention. Administration of the composition as a medicament may be via oral, subcutaneous, direct intravenous, slow intravenous infusion, continuous intravenous infusion, intravenous or epidural patient controlled analgesia (PCA and PCEA), intramuscular, intrathecal, epidural, intracistemal, intraperitoneal, transdermal, topical, buccal, sublingual, transmucosal, inhalation, intra-atricular, intranasal, rectal or ocular routes, abuse deterrent and abuse resistant formulations, sterile solutions suspensions and depots for parenteral use, and the like, administered as immediate release, sustained release, delayed release, controlled release, extended release and the like.

In addition to the aforementioned compounds of the invention, the pharmaceutical composition can contain two or more compounds according to the present invention and also other therapeutically active substances.

The dosage of the pharmaceutical composition according to the present invention can be appropriately selected according to the route of administration, the subject to be administered, the target disease and its severity, age, sex weight, individual differences and disease state. Dosage may be repeated several times a day.

Similar to the above pharmaceutical composition, the term “plant protective composition acceptable carrier or diluent” refers to substances also called co-formulants, which give the plant protective product a form suitable for its application, for example by spraying. They may be a solid or liquid filler, diluent or encapsulating substance which does not interfere with the effectiveness or the biological activity of the active ingredients and which is not substantially toxic to the plants or plant cells to be treated.

In the context of the present invention, pharmaceutically or agrochemically acceptable salts shall mean a salt that can be used in pharmaceutical or plant protective compositions as herein and that possesses the desired pharmacological activity or plant protective activity of the parent compound. The salts also need to be stable and to be usable in the respective compositions. Commonly used salts are hydrochloride, sodium or potassium salts.

Another aspect of the present invention relates to a method for producing the pharmaceutical composition according to the present invention comprising the steps of providing at least one lipopeptide according to the present invention and admixing it with at least one pharmaceutically acceptable carrier and/or excipient. The medicament may be formulated in discrete dosage units and can be prepared by any of the methods well known in the art of pharmacy. The pharmaceutical composition of the present invention can be formulated using methods known in the art to provide rapid, sustained or delayed release of the active ingredient after administration to a mammal.

Similar to the above pharmaceutical composition, the plant protective composition can be formulated as well.

Another aspect of the present invention relates to an antimycotic or antiamoebal or amoebicidal composition, in particular as produced, comprising at least one lipopeptide according to the present invention comprising a supernatant of a culture of a suitable strain of the bacterium Pseudomonas, such as, for example, Pseudomonas sp. QS1027, in particular a sterile filtrate of the supernatant. It was surprisingly found that the supernatant of a culture of the suitable strain of the bacterium Pseudomonas, such as, for example, Pseudomonas sp. QS1027 can be directly used as an effective composition.

Preferred is the antimycotic composition according to the present invention, which is for use as a plant protective composition.

Another aspect of the present invention relates to a method for preventing or inhibiting the growth of a fungus or an amoeba, comprising contacting the fungus or the amoeba with the pharmaceutical composition or the plant protective composition according to the present invention or the antimycotic or antiamoebal composition according to the present invention. Preferred is the method according to the present invention, wherein said fungus is a plant pathogen, such as, for example, B. cinerea, Phakopsora pachyrhizi or Alternaria solani, and wherein preferably said method comprises the step of applying said pharmaceutical composition or the plant protective composition according to the present invention as above or the antimycotic composition according to the present invention as above onto a plant.

Another aspect of the present invention then relates to the medical aspect of the present invention, in particular a method for preventing or treating a fungal or an amoebal infection in a subject in need of said prevention or treatment, comprising administering to said subject an effective amount of the at least one lipopeptide according to the present invention or as produced according to the present invention or the pharmaceutical composition according to the present invention. Preferred is the method according to the present invention, wherein the fungal infection is caused by Candida spp., in particular Candida albicans or Candida auris, Sporobolomyces salmonicolor, or Penicillium notatum or wherein the amoebal infection is caused by Dictyostelium discoideum, Acanthamoeba castellanii, or Acanthamoeba comandoni.

All suitable modes of administration are contemplated according to the invention. Administration of the composition as a medicament may be via oral, subcutaneous, direct intravenous, slow intravenous infusion, continuous intravenous infusion, intravenous or epidural patient controlled analgesia (PCA and PCEA), intramuscular, intrathecal, epidural, intracistemal, intraperitoneal, transdermal, topical, buccal, sublingual, transmucosal, inhalation, intra-atricular, intranasal, rectal or ocular routes, abuse deterrent and abuse resistant formulations, sterile solutions suspensions and depots for parenteral use, and the like, administered as immediate release, sustained release, delayed release, controlled release, extended release and the like.

In the present context the term “about” shall indicate a deviation of +/−10% from the value as given, if not indicated otherwise.

Here, the inventors describe the genomics-based identification of keanumycins A-C. These nonribosomal peptides enable bacteria of the genus Pseudomonas to evade amoebal predation. While being amoebicidal at a nanomolar level, these compounds also exhibit a strong antimycotic activity, in particular against the devastating plant pathogen Botrytis cinerea and they drastically inhibit the infection of Hydrangea macrophylla leaves using only supernatants of Pseudomonas cultures.

The structures of the keanumycins were fully elucidated through a combination of nuclear magnetic resonance, tandem mass spectrometry, and degradation experiments revealing an unprecedented terminal imine motif in keanumycin C extending the family of nonribosomal amino acids by a highly reactive building block. In addition, chemical synthesis unveiled the absolute configuration of the unusual dihydroxylated fatty acid of keanumycin A, which has not yet been reported for this lipodepsipeptide class. Finally, a detailed genome-wide microarray analysis of Candida albicans exposed to keanumycin A shed light on the mode-of-action of this potential natural product lead, which will aid the development of new pharmaceutical and agrochemical antifungals.

Notably, amoeba—bacteria predator—prey interactions have been fruitful sources of novel natural products. Amoeba are single-celled eukaryotes that prey on smaller organisms and are ubiquitous in soil and freshwater. Therefore, bacteria sharing the same habitat as amoeba are exposed to a high selection pressure that fosters either evolution of defense mechanism against grazing or coevolution to form a symbiotic bond with the predator. Various amoeba species engage in mutual and antagonistic relationships with bacteria that are mediated by natural products.

Using a combination of nuclear magnetic resonance (NMR), mass spectrometry (MS), degradation experiments, and organic synthesis, the inventors were not only able to elucidate the structure of two new NRLPs but also identify a transient derivative, which shines light on previously unreported biochemical aspects of nonribosomal peptide synthetases (NRPSs). These natural products are powerful pest control agents protecting plants from the devastating fungal phytopathogen Botrytis cinerea, and their antimycotic properties make them good lead structure candidates for the development of antifungal drugs.

Taken together, the inventor's findings show the potential of discovering new natural products in microbial ecological niches that harbor unexplored biochemistry and unprecedented bioactivity, which will help to combat the anti-infective crisis not only from the pharmacological but also from the agricultural side.

The present invention relates to the following items:

Item 1: A lipopeptide according to the following general formula 1,

(Formula 1)
X-L-Ser-D-Dab-Gly-D-Hse-L-Dab-L-allo-Thr-Dhb-L-
Asp-L-Thr

wherein
X is selected from CH₃—(CH₂)₁₄—CONH, CH₃—(CH₂)₁₂—CHOH—CH₂—CONH, and CH₃—(CH₂)₁₁—CHOH—CHOH—CH₂—CONH,
and
wherein the molecule optionally forms a cycle between the L-Ser and the last L-Thr moiety, and pharmaceutically or agrochemically acceptable salts thereof.

Item 2. The lipopeptide according to Item 1, wherein at least one of the L-Thr is replaced by 4-Cl-L-Thr, and/or wherein the L-Asp is replaced by L-β-threo-OH-Asp.

Item 3. The lipopeptide according to Item 1 or 2, selected from the group consisting of

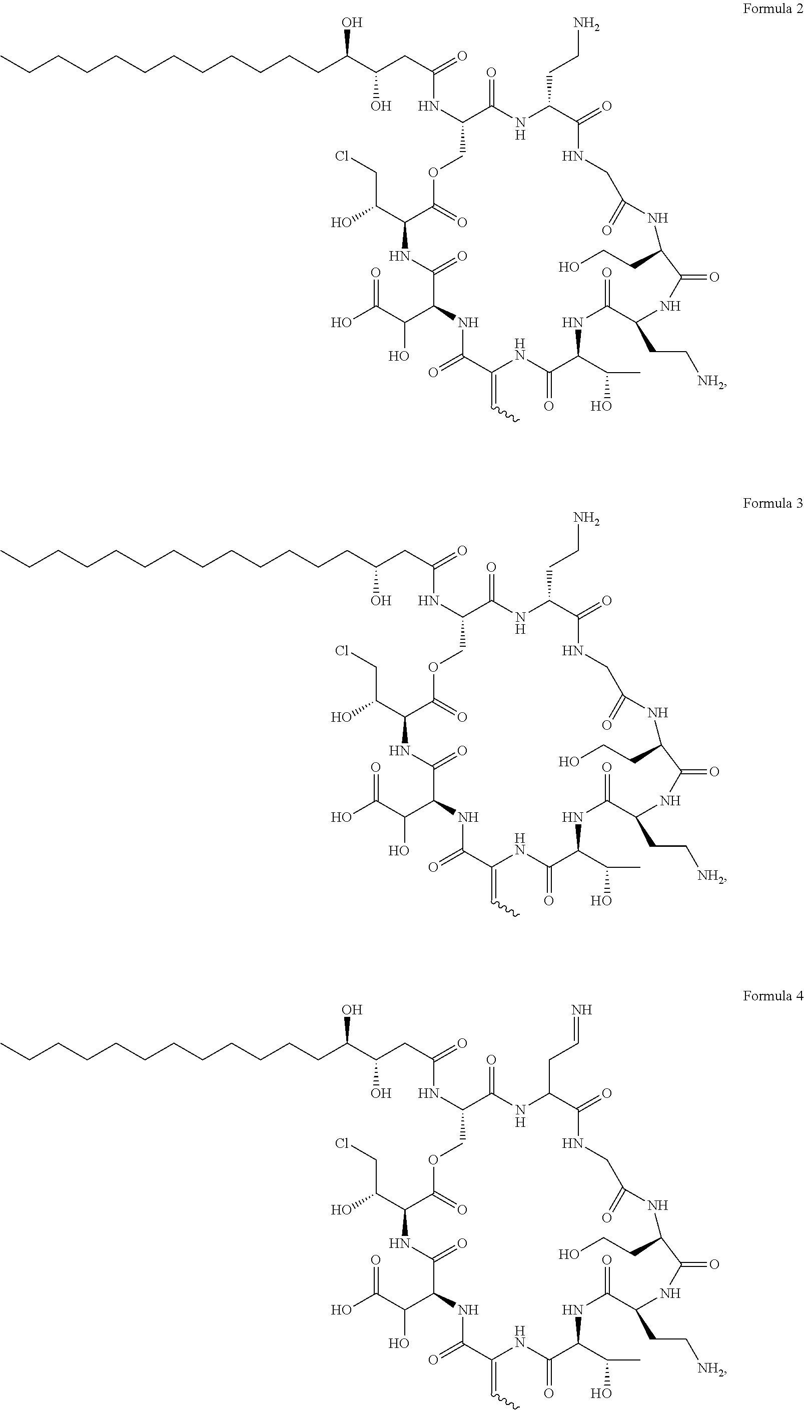

and pharmaceutically acceptable salts thereof.

Item 4. A method for producing the lipopeptide according to any one of Items 1 to 3, comprising
a) suitably culturing a suitable strain of the bacterium Pseudomonas, such as, for example,
Pseudomonas sp. QS1027,
b) extracting the supernatant of the culture with an organic solvent, such as, for example, n-butanol,
c) dissolving the dried extract of b) in alcohol, such as, for example, MeOH,
d) fractioning the extract of c) using reverse phase chromatography and elution with 75% (v/v) acetonitrile in water containing 0.1% (v/v) formic acid, and
e) final purification using preparative HPLC.

Item 5. A method for producing the lipopeptide according to any one of Items 1 to 3, comprising chemical synthesis of the fatty acid chain and the amino acid chain comprising use of the synthetic amino acid building block 2-amino-4-iminobutanoic acid.

Item 6. A pharmaceutical composition or a plant protective composition comprising at least one lipopeptide according to any one of Items 1 to 3 or as produced according to Items 4 or 5, together with at least one pharmaceutically acceptable carrier or diluent, or at least one plant protective composition acceptable carrier or diluent.

Item 7. An antimycotic or antiamoebal composition comprising at least one lipopeptide according to any one of Items 1 to 3 comprising a supernatant of a culture of a suitable strain of the bacterium Pseudomonas, such as, for example, Pseudomonas sp. QS1027, in particular a sterile filtrate of the supernatant.

Item 8. The pharmaceutical composition or a plant protective composition according to Item 6 or the antimycotic composition according to Item 7, which is for use as a plant protective composition, for example in a crop plant, such as soy bean.

Item 9. A method for preventing or inhibiting the growth of a fungus or an amoeba, comprising contacting the fungus or the amoeba with the pharmaceutical composition or the plant protective composition according to Item 6 or the antimycotic or antiamoebal composition according to Item 7 or 8.

Item 10. The method according to Item 9, wherein said fungus is a plant pathogen, such as, for example, B. cinerea, Phakopsora pachyrhizi or Alternaria solani, and wherein preferably said method comprises the step of applying said pharmaceutical composition or the plant protective composition according to Item 6 or the antimycotic composition according to Item 7 or 8 onto a plant.

Item 11. The method according to Item 9 or 10, wherein the concentration of the lipopeptide in the pharmaceutical composition or the plant protective composition according to Item 6 or the antimycotic or antiamoebal composition according to Item 7 or 8 is in a range of about 0.05 μM to about 1.5 μM, preferably about 0.05 μM to about 1 μM, and most preferably at about 0.1 μM.

Item 12. A method for preventing or treating a fungal or an amoebal infection in a subject in need of said prevention or treatment, comprising administering to said subject an effective amount of the at least one lipopeptide according to any one of Items 1 to 3 or as produced according to Items 4 or 5 or the pharmaceutical composition according to Item 6.

Item 13. The method according to Item 12, wherein the fungal infection is caused by Candida spp., in particular Candida albicans or Candida auris, Sporobolomyces salmonicolor, or Penicillium notatum or wherein the amoebal infection is caused by Dictyostelium discoideum, Acanthamoeba castellanii, or Acanthamoeba comandoni.

### BRIEF DESCRIPTION OF THE FIGURES

The invention will now be described further in the following examples with reference to the accompanying figures, nevertheless, without being limited thereto. For the purposes of the present invention, all references as cited are incorporated by reference in their entireties.

FIGS. 1A-1B show that Pseudomonas sp. QS1027 produces keanumycins. (1A) Structure of keanumycins A-C. Main differences between keanumycin A-B and keanumycin C are highlighted in red. (1B) Comparison of edibility using D. discoideum as a predator. Pseudomonas sp. QS1027 WT and corresponding mutants, which lack the ability to produce different natural products, were tested. K. aerogenes served as a positive control.

FIGS. 2A-2D show the structure elucidation of keanumycin A and B. (2A) Key COSY and HMBC correlations observed during NMR analysis of keanumycin A. (2B) Fragmentation pattern of keanumycin A and B as well as of their corresponding degradation products 1 and 2. (2C) GC-MS traces of derivatized fatty acid moiety of keanumycin B and traces of the corresponding synthetic (S)- and (R)-configured 3-hydroxyhexadecanoic acid derivatives 3 and 4. Relative abundances of the extracted-ion chromatogram representing a diagnostic fragment (m/z 237) are shown. (2D) Selected sections of the 1H-NMR spectrum (500 MHz, CDCl₃) of the two carboxylic acid standards 5 and 6 and the isolated fatty acid of keanumycin A.

FIGS. 3A-3D show the structure elucidation of keanumycin C. (3A) Degradation experiments of keanumycin C. Functional groups are highlighted in gray to clarify chemical transformations after degradation experiments. (3B) Comparison of a section of liquid chromatography-MS traces of Marfey's reagent derivatized amino acids derived from keanumycin A and C. (3C) Fragmentation pattern of keanumycin C. (3D) Fragmentation pattern of aldehyde 9.

FIGS. 4A-4C show the ability of Pseudomonas sp. QS1027 fermentation broth to inhibit Botrytis blight. (4A) Representative images of detached H. macrophylla leaves infected with mycelium plugs of B. cinerea 3- and 5-days post infection (dpi). Control medium contains no keanumycin A, whereas supernatant (Sup.) 1 and 2 contain 0.8 and 2.8 mg L-1 keanumycin A, respectively. (4B and 4C) Show graphs representing the lesion area induced by B. cinerea after 3 and 5 dpi. The mean with standard deviation is presented. Single-factor analysis of variance was performed (Tukey-HSD-test and Bonferroni correction) to evaluate statistical significances between different groups (control, Sup. 1, Sup. 2) at a ***p<0.001 and **p<0.01≤ p<0.01).

FIG. 5 shows pathways enriched in C. albicans upon treatment with keanumycin A. Global short-term transcriptional response of C. albicans to a 1-h sublethal dose exposure of keanumycin A. The graph depicts KEGG pathways (y-axis), that manually curated maps of networks of cellular processes, that were significantly enriched and over-represented in a gene set enrichment analysis of genes 2-fold up or down regulated (vs untreated). Abbreviation: KEGG, Kyoto Encyclopedia of Genes and Genomes.

FIG. 6 shows microscopy images of C. albicans incubated with 1% DMSO containing either 4 μg mL-1 keanumycin A or tunicamycin. Keanumycin A permeabilizes the cell wall of the fungi as can be seen by the green fluorescent stain of SYTOX Green. In contrast, also tunicamycin was used at the MIC, the membrane integrity of C. albicans is not disturbed by this natural product. Only isolated cells are stained, which can be explained by the fact that the dye can also be used to distinguish dead from live cells. Scale bar=100 μm.

FIGS. 7A-7B show in 7A) Keanumycin A protected human vaginal epithelial cells in C. albicans infection model. Micrograph depicts % damage to human vaginal epithelial cells A431 (y-axis) in the presence of keanumycin A at varied concentrations (x-axis) as estimated by LDH cytotoxicity assay after 24 hours of infection or treatment. LDH absorbance was normalized to C. albicans infected and uninfected controls. Keanumycin A protected vaginal cells from damage by C. albicans at a narrow concentration range from 4-8 μg/ml (black line). Keanumycin A was toxic to vaginal cells at concentrations above 8 μg/ml (grey line) (N=3). 7B) Keanumycin A reduced C. albicans viability in an infection model. Micrograph depicts % fungal viability (y-axis) of C. albicans in a vaginal epithelial infection model which was treated with varied concentrations of keanumycin A (x-axis). Vaginal epithelial cells were lysed after 24-hour infection, and fungal viability was measured by XTT absorbance, normalized to C. albicans infected and uninfected controls (N=2)

FIG. 8 shows photos of co-culture plates of Pseudomonas sp. QS1027 and Botrytis sp. BcHyd21.

FIGS. 9A-9B show the proposed hypothetical biosynthesis pathway of keanumycin A and B.

FIG. 10 shows the effect of keanumycin against soybean rust (SBR) as a preferred example for crop plants. Application of keanumycin on soybean leaves at concentrations of 1 μM and 0.1 μM were protective, at least in part. Furthermore, no phototoxic effects could be identified. POS=synthetic fungicide (control). The plot shows (from left to right): 24.7%, 11.7%, 12.9%, and 2.1% of leaf areas with symptoms, respectively.

### EXAMPLES

### Materials and Methods

### Bioinformatics and Genome Analysis

The genome of Pseudomonas sp. QS1027 (GenBank Accession Number PHSU01000000) was analyzed with the online version of antiSMASH version 5.2.0. 1 Two contigs were identified that contain two NRPS genes (contig 19; keaA and keaB) and multiple tailoring enzyme genes (contig 13; keaC, keaD, keaE, keaF, and keaG), which showed a high similarity with the nunamycin/syringomycin BGC according to the KnownClusterBlast analysis that is also part of the antiSMASH analysis.

### General Molecular Biology and Microbiology Methods

All aqueous solutions were prepared with Millipore water. Liquid and solid media (1.5% Agar if not stated otherwise, Carl Roth) were autoclaved prior to use (121° C., 15 min). Axenic Dictyostelium discoideum AX2 cells (dictybase.org) were cultured in HL5 liquid medium (Formedium™) and maintained according to previously published procedures. LB (Carl Roth) and SM5 (Formedium™) were prepared in accordance with the manufacturer instructions. Cultures of Escherichia coli strains were incubated at 37° C. and Pseudomonas strains at 28° C. if not stated otherwise. 5 mL liquid cultures were shaken at 180 rpm. Cryostocks of the bacteria were prepared from 1 mL overnight liquid culture and 500 μL 60% glycerol solution and stored at −80° C. Primers for Gibson assembly were designed using: http://nebuilder.neb.com, synthesized by Thermo Fisher, and dissolved in water to obtain a 100 μM solution. For PCRs a 1:10 dilution of the primer stocks was used. A standard 10 μL reaction mixture contained 5 μL DreamTaq Master mix (Thermo Fisher), 1 μL of diluted primers, 2-30 ng DNA template and water. The PCR was carried out in the ThermoCycler peqSTAR 2X using the indicated methods. PCR products were analyzed using 1% agarose gels stained with MidoriGreen Advance DNA Stain (NIPPON Genetics).

### Plaque Assay

Overnight cultures of the Pseudomonas sp. QS1027, the corresponding 9 mutants and Klebsiella pneumoniae were grown in SM5 broth. D. discoideum AX2 liquid cultures were grown to confluency in liquid HL5 medium and were collected from plates by gentle pipetting. Amoeba cells were washed (200 g, 2 min, 4° C.) with KK2 buffer (2.2 g/L KH₂PO₄ and 0.7 g/L K₂HPO₄), before being adjusted to a concentration of approximately 10.000 cells per μL in KK2 buffer. The assay was carried out on SM5 agar (1.5%) in 12 well plates. 20 μL of the bacterial overnight cultures were spread on each well and were dried for 10 minutes. Afterwards 2 μL of the AX2 culture (15,000 to 20,000 cells) were spotted in the middle of each well. The wells were dried for approximately 2 min and the plate was incubated at 22° C. in a box with high relative humidity for 6-7 days. The wells were then examined for fruiting body formation of D. discoideum AX2. K. pneumoniae served as a positive and Pseudomonas sp. QS1027 (WT) as a negative control. The assay was performed in biological duplicates of technical triplicates.

### Metabolic Profiling of Mutants Via LC/MS

Cultures of the respective bacterial strain were inoculated from cryo stocks in 10 mL LB medium and cultivated in 50 mL Erlenmeyer flasks on a gyratory shaker (180 rpm) at 22° C. for 24 h. Approximately 500 mg Amberlite® XAD4 resin (previously washed with water, MeOH, and acetone) was added per 10 mL culture and shaken at 180 rpm at 22° C. for 30 min. The mixture was strained using a cell strainer (40 μm, Sarstedt). The remaining resin was washed with 15 mL water and was extracted with 35 mL MeOH. The solvent was removed in vacuo and the residue was dissolved in 200 μL MeOH, filtered through a 0.2 μm PTFE syringe filter (VWR), and further analyzed via LC-MS by injecting 20 μL of the corresponding sample. LC-MS analysis was performed on a Shimadzu LCMS-2020 equipped with an ESI ion source and quadrupole mass analyzer using a Kinetex® 1.7 μm C18 (100 Å, 50×2.1 mm, Phenomenex) column. Column oven was set to 40° C.; scan range of MS was set to m/z 150 to 2,000 with a scan speed of 10,000 u/s and event time of 0.25 s under positive and negative mode. Desolvation line temperature was set to 250° C. with an interface temperature of 350° C. and a heat block temperature of 400° C. The nebulizing gas flow was set to 1.5 L/min and dry gas flow to 15 L/min. If not otherwise stated, the following method was used: flow rate of 0.7 mL per minute; 0-0.5 min: 10% (v/v) acetonitrile in water containing 0.1% formic acid; 0.5-8.5 min: linear gradient from 10% to 100% (v/v) acetonitrile in water containing 0.1% formic acid; 8.5-11.5 min: 100% acetonitrile containing 0.1% formic acid.

### Regulation of Keanumycin Production Via LC/MS

100 μL overnight cultures of the respective bacterial strain were used to inoculate 10 mL LB medium, which was cultivated in 50 mL Erlenmeyer flasks on a gyratory shaker (180 rpm) at 22° C. for 7 h. 50 μL DMSO was added to the WT, DjesR1, DjesR2, and one of the two Djes1 cultures. N-Hexanoyl-D/L-homoserine lactone (C6-AHL, 4 mg, Sigma Aldrich) dissolved in 50 μL DMSO was added to the second DjesI culture. The cultures were cultivated for additionally 17 h and extracted and analyzed in the same manner as mentioned above.

### Large Scale Fermentation of the Triple Gene Deletion Mutant DvifDjesDmup and Isolation of Keanumycins

20 L 10× Minimal Davis Medium (per liter: 18.4 g glycerol, 174 mg dipotassium phosphate (K₂HPO₄), 5 g ammonium sulfate, 3.25 g trisodium citrate (dihydrate), 580 mg sodium chloride, 820 μL of a 1 M magnesium sulfate solution, 1 mL of a 0.1 M iron (III) chloride solution containing 1% hydrochloric acid) were inoculated with 200 mL (1%) of an overnight culture (22° C.) of the triple gene deletion mutant DvifDjesDmup (created in analogy to previously published procedures) in 10× Minimal Davis Medium. The culture was fermented for 48 h at 25° C. in a continuous stirred-tank reactor. 400 g Amberlite® XAD4 resin (20 g per liter) was added to the culture and stirred for 30 min. The culture was filtered, and the resin was washed with 5 L water. Adsorbed compounds were eluted with 30%, 50% and 70% MeOH in water and pure MeOH (5 L per fraction). Every fraction was analyzed for the presence of keanumycins using an LC/MS standard method. The 100% MeOH fraction contained the highest keanumycin level and was evaporated to dryness in vacuo. The 100% MeOH fraction was further fractioned by using a reverse phase cartridge (C18, 12 g, Biotage) on the flash purification system Isolera™ Prime (Biotage). The extract was eluted with 100 mL of 15%, 30%, 50% and 75% (v/v) acetonitrile in water and 100% acetonitrile (all solvents with 0.1% (v/v) formic acid) applying a flow of 15 mL/minute. Every fraction was analyzed for the presence of Keanumycins using the LC/MS standard method. The 50% acetonitrile fraction was further fractioned by size exclusion chromatography (Sephadex LH-20, 32×1 cm, column volume: 25 mL). Methanol was used as solvent and fractions of 2 mL were collected. Fractions containing the lipopeptide (analyzed via LC/MS standard method) were combined and concentrated in vacuo. Keanumycins were finally purified using preparative HPLC (HPLC column Luna® 5 μm C18 (2), 100 Å, 250×21.2 mm). The following method was used: flow rate of 20 mL per minute; 0-2.5 min: 20% (v/v) acetonitrile in water containing 0.1% formic acid; 2.5-25 min: linear gradient from 20% to 50% (v/v) acetonitrile in water containing 0.1% formic acid; 25-27 min: linear gradient from 50% to 100% (v/v) acetonitrile in water containing 0.1% formic acid; 27-37 min: 100% acetonitrile containing 0.1% formic acid. The retention times (tR) of the compounds keanumycin A and B were tR=19.5 min (2.8 mg) and tR=21.6 min (2.1 mg, not pure; impurity tR=21.4 min), respectively.

For the isolation of keanumycin C, 20 L modified Minimal Davis Medium (per liter: 2 g glycerol, 174 mg dipotassium phosphate (K₂HPO₄), 5 g ammonium sulfate, 9 g trisodium citrate (dihydrate), 580 mg sodium chloride, 820 μL of a 1 M magnesium sulfate solution, 1 mL of a 0.1 M iron (III) chloride solution containing 1% hydrochloric acid; pH=6.9 after sterilization) were inoculated with 200 mL (1%) of an overnight shaking culture (22° C.) of the triple gene deletion mutant DvifDjesDmup in LB Medium. The culture was fermented for 26 h maintaining 22° C., 2.5 slpm aeration and variable stirring (200-250 rpm to keep the pO2 value @ at least 20%) in a continuous stirred-tank reactor. The pH was regulated to 7.2 using 20% (v/v) sulfuric acid in water. The culture was centrifuged (15 min at 6.000 g at 4° C.) and extracted with 20 L n-butanol. The organic phase was evaporated to dryness to yield 9 g of residue. The residue was dissolved in 150 mL MeOH and loaded on ISOLUTE®. The extract was further fractioned by using a reverse phase cartridge (C18, 60 g, Biotage) on the flash purification system Isolera™ Prime (Biotage). The extract was fractioned by elution of 550 mL fractions of 15%, 30%, and 50% (v/v) acetonitrile in water (without formic acid) applying a flow of 40 mL/min. Afterwards 550 mL fractions of 75% (v/v) acetonitrile in water and 100% acetonitrile containing 0.1% (v/v) formic acid were collected applying a flow of 40 ml/min. The 75% (v/v) acetonitrile in water fraction (containing 0.1% (v/v) formic acid) contained keanumycins A, B, and C. Keanumycins were finally purified using preparative HPLC (HPLC column Luna® 5 μm C18 (2), 100 Å, 250×21.2 mm). The following method was used: flow rate of 20 mL per minute; 0-2.5 min: 20% (v/v) acetonitrile in water containing 0.1% formic acid; 2.5-25 min: linear gradient from 20% to 50% (v/v) acetonitrile in water containing 0.1% formic acid; 25-27 min: linear gradient from 50% to 100% (v/v) acetonitrile in water containing 0.1% formic acid; 27-37 min: 100% acetonitrile containing 0.1% formic acid. The retention time of keanumycin C was tR=18.3 min (˜ 1 mg). The yield of keanumycin A and impure keanumycin B were 4 mg and 2 mg, respectively. Figure S11: Section of the preparative HPLC elugram of the 75% (v/v) acetonitrile in water containing 0.1% (v/v) formic acid fraction. Fraction 8=keanumycin C; Fraction 11=keanumycin A; Fraction 16-17=keanumycin B.

### HRMS and HRMS2 of Keanumycins

High-resolution mass (HRMS) and tandem mass (HRMS2) spectrometry data of the pure natural products were obtained by using a LC-ESI-HRMS system (Accela UPLC system by Thermo Scientific) equipped with an Accucore C18 column (100×2.1 mm, particle size 2.6 μm) coupled with a QExactive Orbitrap mass spectrometer (Thermo Scientific) with an electrospray ionization (ESI) source (for MS2 experiments HCD was set to 20, 25, or 30%). MS data was visualized using Xcalibur and mMass. Chemical structures were drawn using ChemDraw and m/z values were calculated with the same software. Most fragment ions could be identified and showed only small deviations from the calculated m/z ratios (D<5 ppm).

### NMR Analysis of Keanumycin A

For NMR experiments keanumycin A (2.8 mg) was dissolved in 200 μL d3-methanol (Deutero GmbH) and 1 H—and 13C-NMR as well as 2D-NMR experiments (COSY, HSQC, HMBC, TOCSY and NOESY) were recorded on a Bruker Avance™ III 600 using standard pulse sequences. Topspin 3.2 (Bruker) was used for the analysis of the NMR data. The solvent peak of d3-MeOH was used for calibration (49.00 ppm for 13C spectra and 3.31 ppm for 1 H-NMR spectra). The signal fine structures are described, using the following abbreviations: s (singlet), d (doublet), t (triplet), q (quartet) and m (multiplet) as well as combinations thereof.

### Marfey's Analysis of Keanumycins

200 μg of keanumycin A, approximately 100 μg keanumycin B, and approximately 250 μg keanumycin C were hydrolyzed by adding 200 μL 6 N HCl and heating to 100° C. for 14 hours. HCl and water were removed in vacuo and the residue was dissolved in 100 μL water. For comparison, aqueous solutions of amino acid standards were prepared (0.25 μmol of each amino acid in 100 μL water). The 3-OH-L/D-threo-aspartate standard was synthesized according to known literature procedures. A protected 4-chloro-L-threonine (NHCbz and COOBn protected) was synthesized according to a literature protocol. The protected 4-chloro-L-threonine derivative was deprotected by suspending 1 mg of the compound in 100 μL 6 N HCl. The suspension was heated to 80° C. for 8 hours. Subsequently, the solvent was removed in vacuo and the product (4-chloro-L-threonine) was used as a standard in the Marfey's analysis. The other amino acid standards were purchased from a commercial vendor (TCI). To 100 μL of the sample and standard solutions, 200 μL of 1% 1-fluoro-2-4-dinitrophenyl-5-Lalanine amide (L-FDAA) in acetone were added. The reaction was started by addition of 40 μL 1 M NaHCO₃ solution and incubated for one hour at 40° C. and 450 rpm. The reaction was quenched by addition of 25 μL 2 N HCl solution. The L-FDAA modified amino acids and the sample were analyzed (5-10 μL injection volume) by LC-MS using a Luna® 5 μm C18 (2) (100 Å, 150×2 mm, Phenomenex) column. The following method with a flow rate of 0.2 mL per minute was used: 0-4 min: 10% (v/v) acetonitrile in water containing 0.1% formic acid; 4-19 min: linear gradient from 10% to 100% (v/v) acetonitrile in water containing 0.1% formic acid; 19-29 min: 100% acetonitrile containing 0.1% formic acid. The L-FDAA modified amino acids and the sample were additionally analyzed (25 μL injection volume) by analytic HPLC using a Luna® 5 μm C18 (2) (100 Å, 250×4.6 mm, Phenomenex) column. The following method with a flow rate of 1 mL per minute was used: 0-2 min: 10% (v/v) acetonitrile in water containing 0.1% formic acid; 2-32 min: linear gradient from 10% to 30% (v/v) acetonitrile in water containing 0.1% formic acid; 32-34 min: linear gradient from 30% to 100% (v/v) acetonitrile in water containing 0.1% formic acid, 34-40 min: 100% acetonitrile containing 0.1% formic acid. The modified amino acids were detected by their specific absorption pattern using a photodiode array detector (2=340 nm) and their m/z ratios. The software LabSolutions Version 5.97 (Shimadzu) was used for HPLC and LC-MS data analysis. The chromatograms of keanumycin B and C were directly compared with the chromatogram of keanumycin C.

### GC-MS Experiments to Decipher the Stereochemistry of the Fatty Acid in Keanumycin B

1.0 mg keanumycin B (0.86 μmol) was stirred in 200 μL 6 N HCl for 18 h at 100° C. After cooling to r.t. the solution was extracted with 3×0.5 mL CHCl₃. The combined organic phases were dried with sodium sulfate and solvents were evaporated under reduced pressure. The crude extract was dissolved in 500 μL anhydrous methanol and trimethylsilyldiazomethane (0.5 M in Ether, 60 μL) was added, giving a yellow color. After stirring for 10 min at r.t., the reaction was quenched via addition of 2 μL formic acid. During quenching the solution turned colorless immediately. 1 mL toluene was added, and solvents were evaporated in vacuo. The residue was dissolved in 500 L anhydrous DCM and 1.0 mg DMAP prior to 2 μL(S)-MTPACl was added. The mixture was stirred at r.t. for 30 min. Then 500 μL 0.1 M HCl was added, and the reaction mixture was extracted with EtOAc (3×1 mL). The combined organic phases were dried with sodium sulfate and solvents were evaporated in vacuo. Finally, the derivative was dissolved in 100 μL CHCl₃ and analyzed via GC-MS. GC-MS analysis was performed on a Trace 1310 GC (Thermo Scientific) coupled with a TSQ 9000 electron impact (EI)-triple quad mass spectrometer (Thermo Scientific). A 4 mm GC inlet glass liner with glass wool and a BPX5 capillary column (30 m, 0.25 mm inner diameter, 0.25 μm film) from SGE was used. The column was operated with helium carrier gas (0.6 mL/min) and split injection (split flow: 15 mL/min, ratio 1:25 for synthetic references and split flow: 5 mL/min, ratio 1:8 for natural product sample). The injector temperature was set to 250° C., the GC temperature was set to 200° C. (isothermic program for 4 h), MS transfer line was set to 300° C., the ion source temperature was set to 200° C. Total ion current (TIC) values were recorded in the mass range of 45-600 amu and the extracted-ion chromatogram (EIC) of the mass m/z=237 was used to illustrate the results

### Amoebicidal Activity of Keanumycin A

Amoebicidal activity of keanumycin A against D. discoideum was determined using a previously published method. In brief, 3000 D. discoideum cells (AX2) were cultured in 96-well plates (Sarstedt) in 200 μL HL5 medium containing 1% DMSO (Carl Roth) and a specified amount of compound (starting from 0.5 μM; 2-fold serial dilution) in triplicates at 22° C. After 72 h the cell concentration was determined according to the manufacturers instruction using a CASY® Cell Counter+Analyser System (Model TT, Roche Innovatis AG) equipped with a 60 μm capillary and the evaluation cursor set to 7.5-17.5 μm. The viable cell concentration (including standard deviation) was plotted against the logarithmic concentration of the compound and the IC50 value was determined using PRISM (GraphPad, Version 5.03). The average IC₅₀ was found to be 4.4 nM (two experiments).

Amoebicidal activity of keanumycin A against Acanthamoeba was determined by culturing A. castellanii or A. comandoni in 96-well plates (Sarstedt) in 100 μL PYG medium (ATCC Medium 712; https://www.atcc.org) containing 1% DMSO (Carl Roth) and a specified amount of compound (starting from 50 μM; 2-fold serial dilution) in triplicates at 28° C. After 96 h the cell viability was determined using a resazurin assay. Therefore, 20 μL of a 1 mM resazurin sodium (Sigma) solution in water was added per single well before it was allowed to incubate for another 3 h at 28° C. Using a plate reader (Tecan Life Sciences, excitation 560 nm and emission 590 nm) the fluorescence emission was determined. The fluorescence (including standard deviation) was plotted against the logarithmic concentration of the compound and the IC₅₀ value was determined using PRISM (GraphPad, Version 5.03). The average IC₅₀ was found to be 3.1 μM against A. comandoni (two experiments), and 2.0 μM against A. castellanii (two experiments), respectively.

### Antiproliferative Effect and Cytotoxicity of Keanumycin A

Keanumycin A was assayed for antiproliferative effects (GI50) using human umbilical vein endothelial cells HUVEC (ATCC CRL-1730) and human chronic myeloid leukemia cells K-562 (DSM ACC 10) and for their cytotoxic effects (CC50) using human cervix carcinoma cells HeLa (DSM ACC 57) as previously described). The test compounds were dissolved in DMSO (10 mg mL-1).

TABLE 1
and CC₅₀ values of keanumycin A. Converted values
are GI₅₀ (23 μM) and CC₅₀ (30 μM)

| Antiproliferative Effect | Antiproliferative Effect | Cytotoxicity |
|---|---|---|
| HUVEC | K-562 | HeLa |
| GI₅₀ [μg/ml] ≥ 50 | GI₅₀ [μg/ml] = 26.8 (±1.2) | CC₅₀ [μg/ml] = 34.9 (±2.0) |

### Antimicrobial Activity of Keanumycin A

A qualitative screen of different microorganisms was performed using a disk diffusion test. Each microorganism was plated on Mueller Hinton agar plates and 9 mm holes were punched out. The respective compounds were dissolved in the indicated solvent to yield solutions of specified concentrations. 50 μL of the respective solutions were placed in each hole. The plates were incubated for 24 h at 37° C. After incubation, the size of inhibition zones was determined.

TABLE 2
Results of the antimicrobial activity assay. Ciprofloxacin (antibiotic)
and amphotericin B (antifungal) served as controls.

| | Inhibition area diameter [mm] | | | |
|---|---|---|---|---|
| | | Ciprofloxacin | Amphotericin B | Keanumycin A |
| | Solvent | (5 μg/mL | (10 μg/mL in | (1 mg/mL in |
| Organism | (Methanol) | in water) | DMSO/MeOH) | MeOH) |
| Bacillus subtilis | 0 | 29 | — | 11 |
| Staphylococcus | 0 | 17 | — | 10 |
| aureus SG 511 | | | | |
| Escherichia coli | 0 | 26 | — | 0 |
| Pseudomonas | 0 | 26 | — | 0 |
| aeruginosa | | | | |
| SG 137 | | | | |
| Pseudomonas | 0 | 28 | — | 0 |
| aeruginosa | | | | |
| K799/61 | | | | |
| Staphylococcus | 0 | 0 | — | 0 |
| aureus | | | | |
| (MRSA) 134/94 | | | | |
| Enterococcus | 0 | 16 | — | 12 |
| faecalis | | | | |
| (VRE) 1528 | | | | |
| Mycobacterium | 0 | 22 | — | 13 |
| vaccae | | | | |
| Sporobolomyces | 10 | — | 18 | 28 |
| salmonicolor | | | | |
| Candida albicans | 0 | — | 21 | 30 |
| Penicillium | 10 | — | 18 | 26 |
| notatum | | | | |

The activity assay was performed using the broth dilution method according to the EUCAST DEFINITIVE DOCUMENT E.DEF 9.3.2. B. cinerea and A. solani were incubated according to the Methods for dilution antimicrobial susceptibility tests for bacteria that grow aerobically by the NCCLS. Keanumycin A was dissolved in DMSO.

TABLE 3
Results of the broth dilution method assay for different human pathogenic
fungi. Fungi were either isolates from clinical patients or received
from the American Type Culture Collection (ATCC).

| | Keanumycin | | | |
|---|---|---|---|---|
| | A | Voriconazol | Posaconazol | Fluconazol |
| Species | in mg/L | in mg/L | in mg/L | in mg/L |
| Candida | 0.5-1 | 1 | 0.06 | <64 |
| auris | | | | |
| 2021-353 | | | | |
| Candida | 1 | 0.06 | 0.25 | 2 |
| glabrata | | | | |
| 2021-359 | | | | |
| Candida | 1 | 0.03 | 0.06 | 2 |
| parapsilosis | | | | |
| ATCC 22019 | | | | |
| Candida | 1 | 1 | 0.125 | 64 |
| tropicalis | | | | |
| 2021-360 | | | | |
| Aspergillus | 4 | 0.5 | 0.125 | n.d. |
| fumigatus | | | | |
| ATCC 204305 | | | | |
| Fusarium | >8 | 8 | >8 | n.d. |
| proliferatum | | | | |
| 2021-361 | | | | |
| Rhizopus | >8 | >8 | 4 | n.d. |
| arrhizus | | | | |
| 2021-366 | | | | |
| Scedosporium | 8 | 1 | >8 | n.d. |
| apiospermum | | | | |
| 2016-079 | | | | |

C. albicans Vaginal Epithelial Infection Model

Immortalized vaginal epithelial cell line A431 were seeded at a density of 1*10⁵ cells/ml, into 96-well plates in RPMI, supplemented with 10% FBS, and incubated for approximately 48-hour, at 37° C. and 5% CO₂.

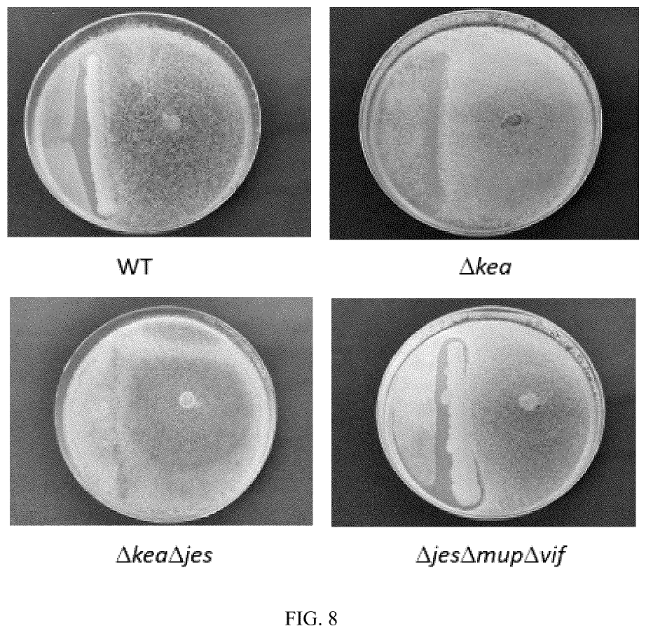

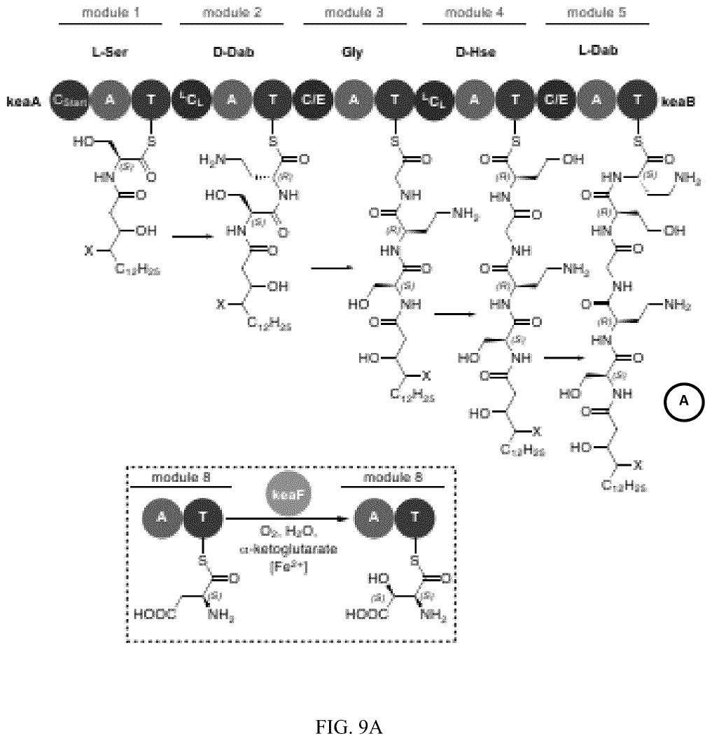

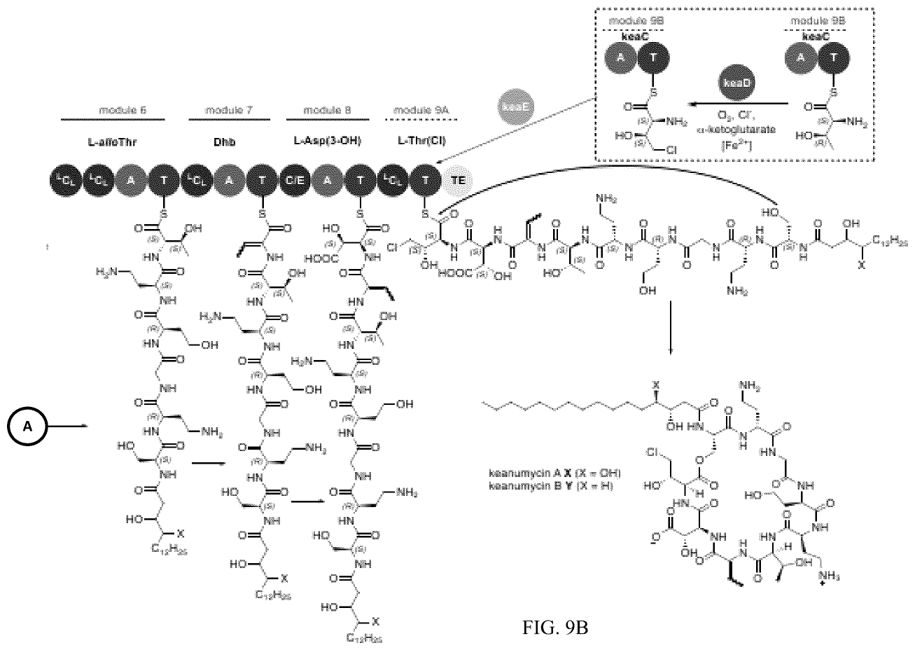

Overnight culture of C. albicans strain SC5314 grown in YPD at 30° C., shaking at 180 RPM was harvested, washed twice with PBS. A suspension of 2*10⁵ cells/ml of yeasts was prepared in fresh RPMI. A drug dilution plate for keanumycin A was prepared for concentrations ranging from 1-16 μg/ml. The yeast suspension or RPMI alone was added to the drug dilution plates, and this suspension was used to replace the medium on confluent cells. Epithelial damage was measured 24 hours after infection by collecting and diluting the supernatant 1:10 in PBS and measuring the lactate dehydrogenase (LDH) activity with the Roche Cytotoxicity Detection Kit (LDH) as per manufacturer's instructions. The absorbance (490 nm) values are represented as percentage damage, which is normalized to the low control, where medium only is 0% damage, and high control, where C. albicans infection is 100% damage. The experiments were performed in technical duplicates, and in two biologically independent replicates. See FIGS. 7A and 7B.

Co-Culture Experiments of Pseudomonas sp. QS1027 and Botrytis sp.

B. cinerea was routinely grown on PDA agar plates at 24° C. A small piece of agar was cut out (5 mm diameter) and used to inoculate a fresh PDA plate. Afterwards, approximately 2-3 cm next to the fungal inoculum either the WT strain, the □kea, the □kea□jes or the □jes□mup□vif mutant was streaked out from a single colony, which had grown on LB agar at 28° C. previously. The co-culture plates were grown for 3 days at 24° C. before pictures were taken (see FIG. 8).

### Detached Leave Assay

Conidia of B. cinerea were collected by flooding 15-20 days old culture plates with sterilized A. dest. For the removal of mycelium fragments the suspension were filtered over a sterilized cheesecloth and the spore suspension were adjusted to 1×10⁵ spores/mL. Small cut pieces of 5 mm-diameter solid GB5+Glucose medium were inoculated with 10 μL of prepared spore suspension and incubated at room temperature for 20 to 24 h for pre-germination. With regard to each treatment, boxes including paper tissues watered with sterile deionized water and two leaves of Hydrangea macrophylla ‘Pink Sensation’ were prepared in duplicates. On each half of the single leaf 50 μL of the sterile filtered DjesDmupDvif mutant supernatant with an addition of one drop of Tween®20 to reduce the surface tension were added and finally, one of the prepared mycelium plugs was transferred directly on the ferment drop. Sterilized liquid LB medium as well mixed with a drop of Tween were used as control treatment. After inoculation the leaves were incubated at 21° C. at 16 h light and 8 h dark. Pictures were taken after three- and five-days post inoculation (dpi) and the lesion area measured by using the program ImageJ 1.53 s. 3 biological replicates consisting of 2 technical replicates were performed. To verify the significance of the different datasets a Tukey- and Bonferroni test were applied, and p-values were calculated by IBM SPSS Statistics 28.0 and Origin 2021 (0.001≤ p<0.01). The concentration of keanumycin A in the different supernatants was calculated by recording a standard curve on an HPLC system and analyzing the supernatants in the same fashion. To generate the standard curve, 40 μL of the standard keanumycin A solutions (1:1 MeOH/water) were injected into a Shimadzu HPLC system (column: Phenomenex Luna C18 (2) (250×4.7 mm); flow rate: 1 mL min-1; 0-1 min: 10% (v/v) acetonitrile in water containing 0.1% formic acid; 1-25 min: linear gradient from 10% to 60% (v/v) acetonitrile in water containing 0.1% formic acid) and the maximal UV absorption @210 nm (retention time of keanumycin A was 19.5-19.7 min) was plotted against the corresponding concentration.

### Results

Bioinformatics Analysis of Pseudomonas sp. QS1027.

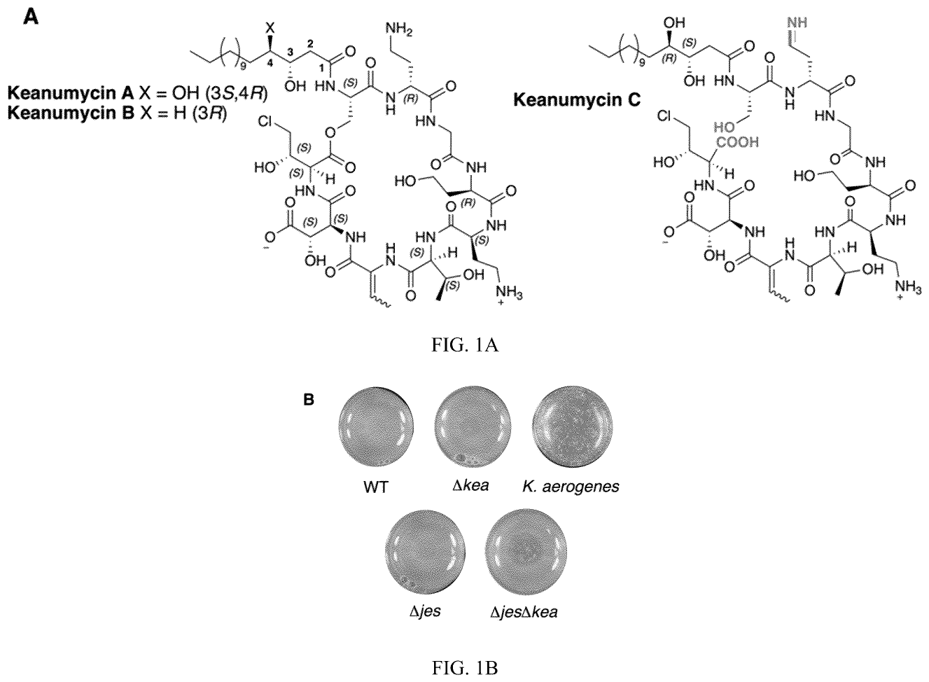

In a previous study, the inventors found that the strain Pseudomonas sp. QS1027, which was isolated from fruiting bodies of the social amoeba D. discoideum, produces the amoebicidal natural product jessenipeptin. Interestingly, the null mutant Δjes, which is not able to produce jessenipeptin, was still toxic for D. discoideum (FIG. 1B). In contrast to the food source Klebsiella aerogenes, which is used for the propagation of D. discoideum, the amoebae were not able to graze on the Δjes strain and to form fruiting bodies. Consequently, the inventors concluded that Pseudomonas sp. QS1027 is able to biosynthesize additional amoebicidal natural products. Mining of the corresponding genome (GenBank accession number PHSU00000000) using antiSMASH revealed multiple biosynthetic gene clusters (BGCs). Manual curation uncovered a BGC that was split between two contigs and displayed high similarity with a nunamycin gene cluster, which is required for the production of antifungal NRLPs belonging to the syringomycin family. Since fungi and social amoebae share many similarities with respect to cellular structures, and many antifungal agents target D. discoideum, the inventors reasoned that this NRPS gene cluster may be responsible for the biosynthesis of another amocbicidal natural product.

To gain insight into the function of this NRPS gene, the inventors first closed the gap between the two contigs by PCR amplification of the intracontig region and subsequent Sanger sequencing of the amplicons. Thus, the inventors obtained the complete NRPS BGC sequence. The resulting contig (GenBank accession number MW331495) shows 100% similarity with the nunamycin BGC. The keanumycin BGC consists of six genes (keaA-F), which code for two NRPSs (KeaA and B) and four enzymes (KeaC-F) involved in the modification of the two C-terminal amino acids (FIG. 1C). KeaA and B contain five and three full modules, respectively, which consist of a condensation (C), adenylation (A), and thiolation (T) domain and select for individual amino acids. KeaB contains a fourth module composed of only a C and a T domain followed by a thioesterase domain, which may catalyze the macrolactonization of the oligopeptide after assembly. The last incomplete module of KeaB is therefore likely loaded by trans-acting enzymes in analogy to the syringomycin biosynthetic machinery (FIG. 1D). KeaC would activate L-Thr via its A domain and would covalently attach the amino acid through a thioester to its T domain. The amino acid would then subsequently be chlorinated by the nonheme Fe2+α-ketoglutarate-dependent enzyme KeaD to generate 4-Cl-LThr, which would then be transferred by the aminoacyltransferase KeaE to the T domain of the last module of KeaB. KeaF probably oxidizes Asp to L-β-threo-OH-Asp.

### Molecular Biological Evaluation of the Kea BGC

To test if the keanumycin BGC allows the production of an amoebicidal compound, the inventors generated an in-frame deletion of the gene fragment coding for the A domain of the second module of keaA resulting in the mutant Δkea (FIG. 1C). The Δkea mutant itself is still toxic; however, D. discoideum is able to graze on the double mutant ΔjesΔkea (FIG. 1B). Interestingly, comparison of the metabolic profiles of an ethyl acetate extract of wild type (WT) and Δkea mutant cultures revealed no major differences and showed no bioactivity against D. discoideum. After realizing that the new natural products could be too hydrophilic and are therefore not extractable with the inventor's standard method, the inventors screened different absorbent resins (Amberlite XAD4 extracts keanumycin A and B) and solvents (n-butanol can extract all keanumycins) to develop a suitable extraction protocol. Using the new method, the inventors were able to see an absence of metabolites in the Δkea mutant compared to the WT strain, which turned out to be the keanumycins. Hence, the combination of genome mining with a functional bioassay was crucial to identify these hydrophilic NRLPs, which, otherwise, would have been overlooked based on bioassay guided fractionation.

Since the keanumycin BGC is adjacent to the jessenipeptin BGC, the inventors hypothesized that both BGCs are regulated by the same LuxI/LuxR-type quorum sensing (QS) system previously described for the biosynthesis of jessenipeptin. Indeed, using a set of regulatory gene knockout mutants and the signaling molecule N-hexanoyl-homoserine lactone, the inventors were able to show that the production of jessenipeptin and the keanumycins is regulated by the same QS system. Furthermore, the production of the virginiafactins, which are NRLPs also produced by this strain, is not under the control of this particular QS system and is not impaired by the in-frame deletion of gene fragments that were used to generate the Δkea and ΔjesΔkea mutants.

### Structure Elucidation of the Keanumycins A and B

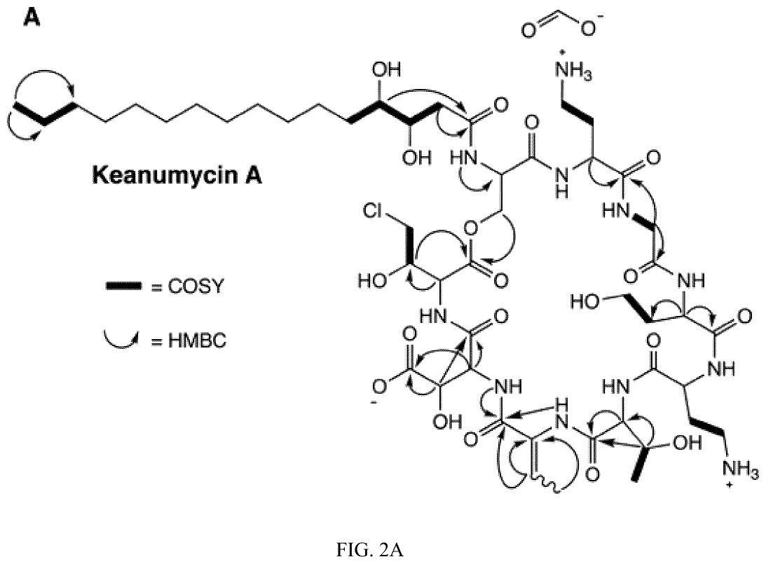

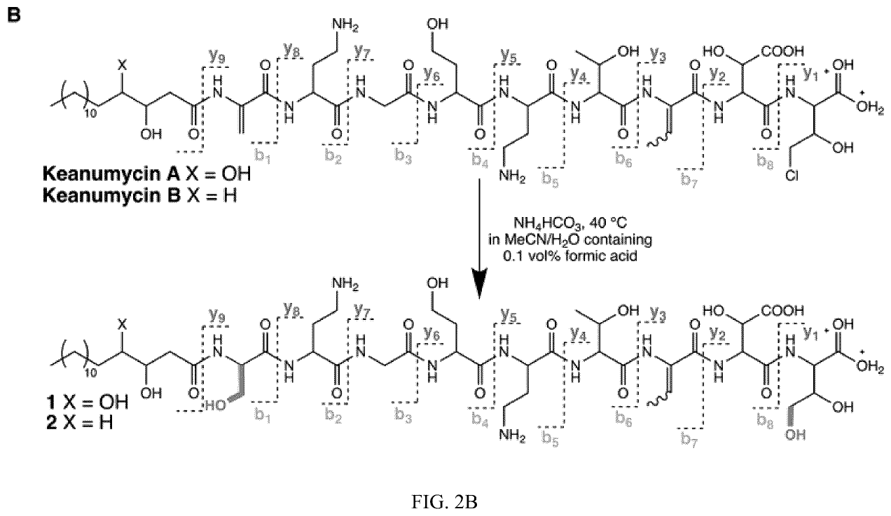

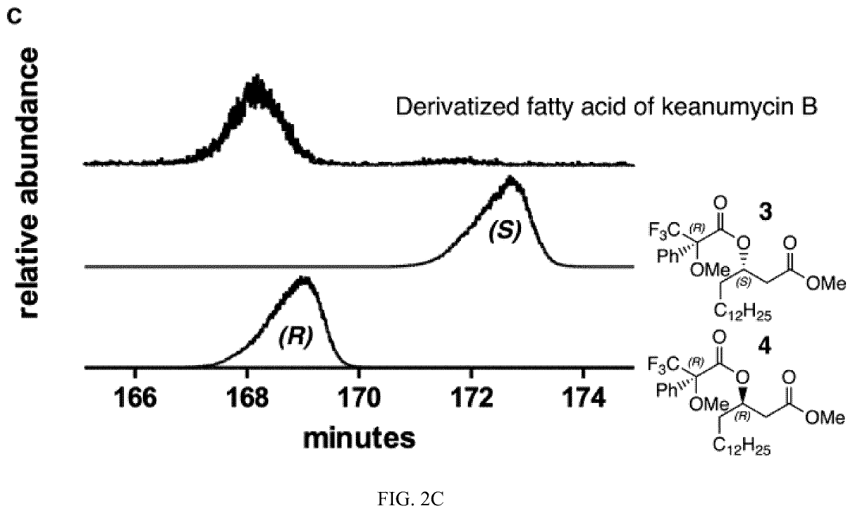

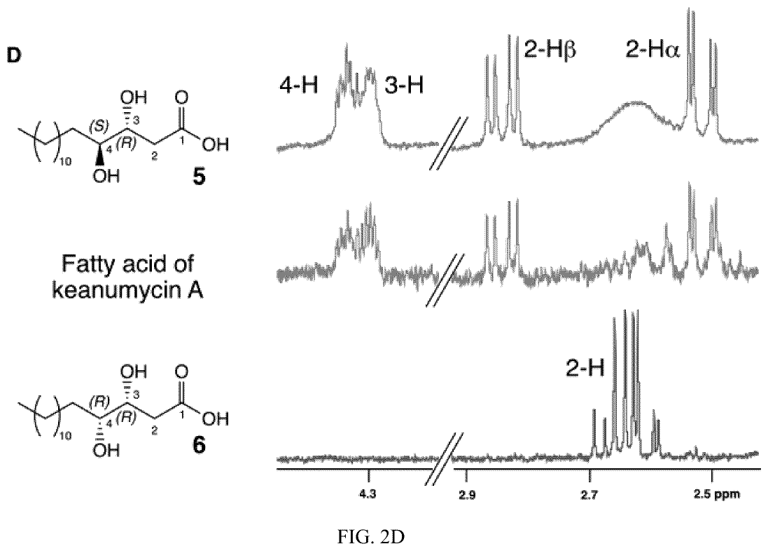

The inventors isolated keanumycin A (m/z 583.7892 [M+2H]2+ consistent with the molecular formula C₄₉H₈₄ClN₁₁O₁₉) and impure keanumycin B (m/z 575.7900 [M+2H]2+; C₄₉H₈₄ClN₁₁O₁₈), which could not be separated from an unknown metabolite (FIG. 1A). The inventors were also able to isolate keanumycin C (m/z 591.7866 [M+2H]2+; C₄₉H₈₄ClN₁₁O₂₀). Unfortunately, keanumycin C slowly degrades in solution or when concentrated to dryness, yielding a poorly soluble solid, which prevented us from recording NMR spectra. Therefore, a detailed NMR analysis was only performed on keanumycin A. Important correlations in the ¹H, ¹H-correlation spectroscopy (COSY) experiment disclosed the presence of a 3,4-dihydroxylated linear fatty acid, which is a rare motif in NRLPs and only described for two other members of the syringomycin family (FIG. 2A). The ¹H,¹³C-heteronuclear multiple bond correlation (HMBC) experiment allowed us to establish a majority of the linear amino acid sequence, the attachment point of the fatty acid and provided evidence for the position of the lactone ring between the carbonyl moiety of the 4-Cl-L-Thr and the alcohol side chain of the Ser. These data were confirmed by high-resolution tandem mass spectrometry (HRMS2) experiments, which showed a fragmentation pattern in accordance with the NMR data and the bioinformatics prediction (FIG. 2B). Saponification of keanumycin A under mild conditions led to the expected dechlorinated and linear lipopeptide 1, whose planar structure was confirmed by further HRMS2 experiments.

Acid-mediated degradation of keanumycin A followed by derivatization of the resulting amino acids with Marfey's reagent and comparison with commercial or synthetic standards established the presence of the following amino acids: 1×Gly, 1×L-Ser, 1×L-Dab, 1×D-Dab, 1×D-Hse, 1×L-allo-Thr, 1×4-Cl-L-Thr, 1×L-β-threo-OH-Asp. With these data in hand, the configurations of all amino acids, with the exception of the L- and D-Dab moieties, are unambiguously determined. Bioinformatics analysis of the keanumycin BGC indicated that the Dab at position two of the peptide chain should be assigned the D-configuration because the third module of keaA is predicted to contain a condensation/epimerization (CE) domain, which not only condenses a Dab with a Gly but also epimerizes the preceding L- to a D-amino acid. In contrast, the Dab at position five should be L-configured, since the first module of KeaB is

predicted to contain a tandem CL domain, which lacks epimerase activity and should not introduce a D-amino acid into the nascent peptide chain. The presence of the tandem CL domain in the NRPS assembly line has been reported for other members of the syringomycin class of cyclic lipopeptides, such as thanamycin or nunamycin. To the inventor's knowledge, the biosynthetic details of the condensation domain duplication have not yet been investigated, and it remains elusive, if one or both domains are crucial for the condensation reaction.

Before focusing on the stereochemistry of the fatty acid of keanumycin A, the inventors first investigated the structure of keanumycin B. The molecular mass difference of 16 Da between keanumycin A and B indicates that both natural products differ by a single oxygen atom. Hence, the inventors concluded that both natural products presumably share the same amino acid sequence but vary in the oxidation state of their fatty acid comparable to pseudomycin A and B. Hydrolysis of keanumycin B followed by derivatization with Marfey's reagent indeed yielded the same amino acid composition as found for keanumycin A. HRMS2 analysis of intact and saponified keanumycin B 2 confirmed the amino acid sequence and hinted toward a monohydroxylated fatty acid connected to the C-terminal Ser, which was further validated by a COSY experiment of the impure keanumycin B (FIG. 2B).

To determine the stereochemistry at the β-position of the fatty acid of keanumycin B, the inventors hydrolyzed the NRLP, isolated the lipid, converted the acid into a methyl ester using trimethylsilyldiazomethane, and esterified the alcohol function using Mosher's acid chloride. The derivatized fatty acid was subjected to gas chromatography (GC)-MS and compared to synthetic standards 3 and 4 (see FIG. 2C). Thus, the inventors established the nature of the fatty acid of keanumycin B to be (R)-3-hydroxyhexadecanoic acid.

With one stereogenic center of the lipid moiety elucidated, the inventors went on to deduce the absolute configuration of the 3,4-dihydroxy moiety of keanumycin A, whose stereochemistry has never been assigned in related compounds (e.g., pseudomycin A and syringostatin B). To this end, the inventors modified a previously published method for the synthesis of the Japanese orange fly lactone. Starting from the chiral pool material D-gluconolactone, the inventors synthesized (3R,4S)- and (3R,4R)-3,4-dihydroxyhexadecanoic acid, 5 and 6, respectively. Comparison of the ¹D ¹H-NMR spectra of 5 and 6 with a sample derived from basic hydrolysis (acidic conditions led to decomposition) of keanumycin A clearly showed that the vicinal system is trans configured. Thus, the inventors concluded that keanumycin A contains a (3S,4R)-3,4-dihydroxyhexadecanoic acid moiety.

### Structure Elucidation of Keanumycin C.

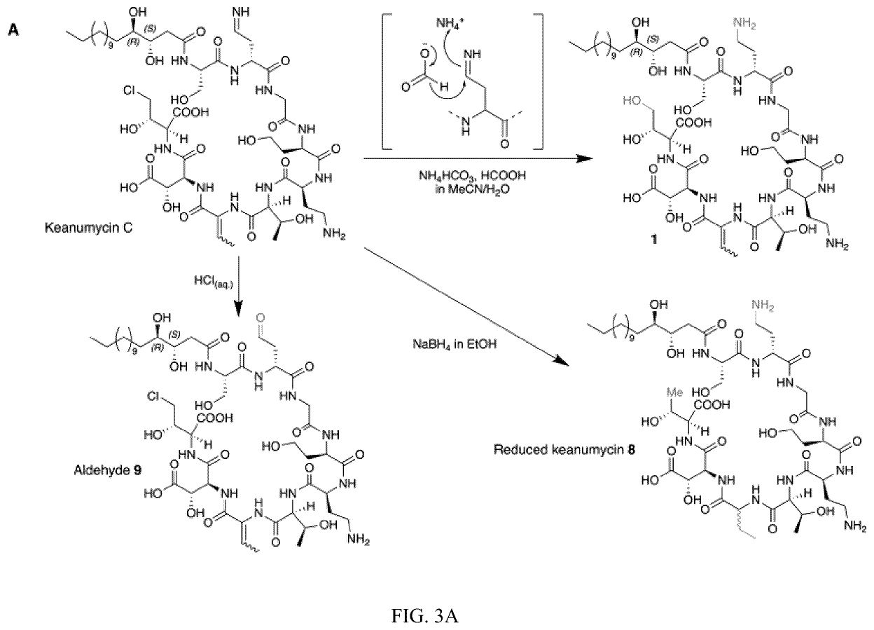

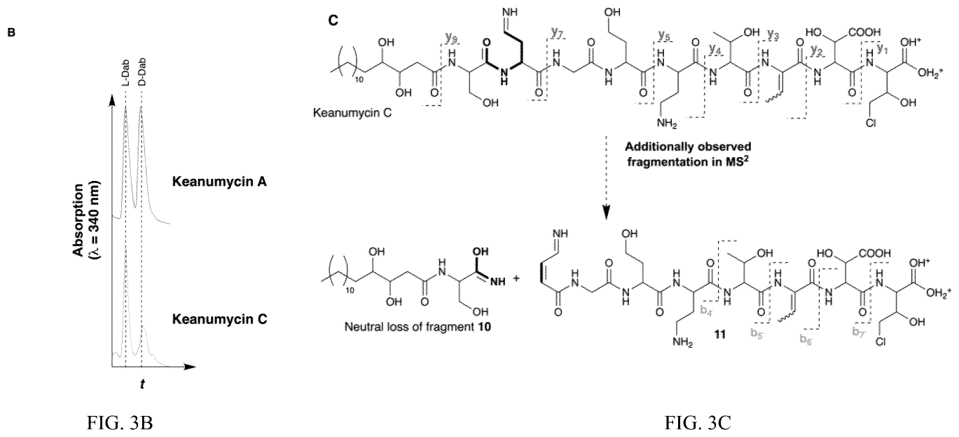

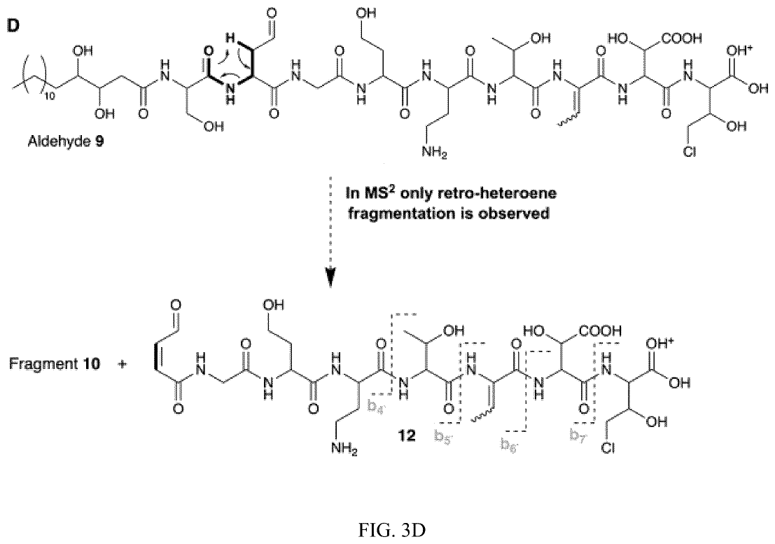

Since keanumycin A and C have a mass difference of 16 Da, the inventors first thought that keanumycin C could bear a trihydroxylated lipid chain. However, after subjecting keanumycin C to various degradation and MS experiments, it became evident that this NRPS product does not contain a macrolactone moiety. Instead, keanumycin C is the first described linear NRLP with a terminal imine moiety. This adds 2-amino-4-iminobutanoic acid to the amino acid building block repertoire, found in NRPs (in this particular case at position 2). Hydrolysis of keanumycin C under mild basic conditions in the presence of formate leads to the linear and dechlorinated degradation product 1, which is the same degradation product obtained after saponification of keanumycin A (FIG. 3A). This observation can only be explained by an Eschweiler-Clarke type reaction using formate as the reducing agent and keanumycin C being a linear lipopeptide. Reduction of keanumycin C with NaBH₄ generated the expected reduced derivative 8. Treatment with diluted HCl led to the formation of the corresponding aldehyde 9. Acidic hydrolysis of keanumycin C followed by derivatization with Marfey's reagent yielded the following amino acid composition: 1×Gly, 1×L-Ser, 1×L-Dab, 1×D-Hse, 1×L-allo-Thr, 1×4-Cl-L-Thr, 1×L-β-threo-OH-Asp. Compared to keanumycin A, keanumycin C lacks 1×D-Dab moiety but generated a derivatized aspartate-4-semialdehyde (FIG. 3B). In contrast, when the reduced compound 8 was subjected to the same derivatization procedure 1×L-Dab and 1×D-Dab could be detected. HRMS2 experiments of keanumycin C further validated the presence of an imine moiety as the fragmentation pattern distinctly differs from keanumycin A and B. Two y fragments (y8 and y6) and all b fragments were absent in the spectrum of keanumycin C when using the previously applied conditions (FIG. 3C). Furthermore, a retro-heteroene reaction leading to the neutral loss of fragment 10 was observed. The remaining ion 11 fragmented further into prominent b′ fragments. Aldehyde 9 yielded an even more unique fragmentation pattern, as no conventional b or y fragments were detected (FIG. 3D). The retro-heteroene reaction was dominant and exclusively yielded the main fragment 12, which further fragmented in analogy to ion 11. Finally, these findings also confirm the predicted absolute configuration of the two Dabs, because the imine at position two results in the absence of the D-Dab during amino acid composition analysis of keanumycin C using Marfey's method.

### Bioactivity of Keanumycin A

The antimicrobial activity of keanumycin A was first tested against D. discoideum (IC₅₀=4.4 nM) and two human pathogenic acanthamoeba (A. castellanii IC₅₀=2.0 μM and A. comandoni IC₅₀=3.1 μM). Keanumycin A proved to be strongly amoebicidal, especially against the amoeba from which the producer strain was isolated. To further explore the antimicrobial potential of the keanumycins, a qualitative screen of different microorganisms was performed using a disk diffusion assay. Keanumycin A is weakly active against Gram-positive bacteria (Bacillus subtilis, Enterococcus faecalis, and Mycobacterium vaccae) and shows no activity against the Gram-negative bacterium Pseudomonas aeruginosa but strongly inhibits the growth of multiple fungi (Sporobolomyces salmonicolor, Candida albicans, and Penicillium notatum), which is a property most members of the syringomycin family share. Encouraged by these results, the inventors determined minimum inhibitory concentrations (MICs) for keanumycin A against different human pathogenic fungi and fungal phytopathogens (Table 4).

TABLE 4
MIC Values of Keanumycin A against Different Human
Fungal Pathogens and Fungal Phytopathogens^{a}

| fungal species strain | MIC [μM] |
|---|---|
| Candida auris NRZ-2021-353 | 0.86 (1 mg L−1) |
| Candida glabrata NRZ-2021-359 | 0.86 (1 mg L−1) |
| Candida parapsilosis ATCC 22019 | 0.86 (1 mg L−1) |
| Candida tropicalis NRZ-2021-360 | 0.86 (1 mg L−1) |
| Aspergillus fumigatus ATCC 204305 | 3.42 (4 mg L−1) |
| Rhizopus arrhizus NRZ-2021-366 | >6.85 (>8 mg L−1) |
| Scedosporium apiospermum NRZ-2016-079 | 6.85 (8 mg L−1) |
| Fusarium annulatum NRZ-2021-361 | >6.85 (>8 mg L−1) |
| Botrytis cinerea SF011406 | 0.07 (80 μg L−1) |
| Alternaria solani SF003858 | 1.07 (1.25 mg L−1) |

^{a}All strains, except for the B. cinerea and A. solani strain, were incubated according to the procedure of The European Committee on Antimicrobial Susceptibility Testing (EUCAST, 35° C.). The B. cinerea and A. solani strains were incubated according to the procedure of the National Committee for Clinical Laboratory Standards (NCCLS, 22° C.).

Keanumycin A is active at low concentrations (0.9 μM or 1 mg L-1) against all Candida spp. tested (Table 4). The natural product was even able to inhibit the proliferation of a clinical isolate of Candida auris, which is resistant against the antimycotic drug fluconazole. In combination with the comparatively low antiproliferative activity (HUVEC: GI₅₀>43 μM and K-562, GI₅₀=23 μM) as well as cytotoxicity (HeLa, CC₅₀=30 μM) and keanumycin A being able to protect human epithelial cells in an in vitro infection model from C. albicans induced damage (FIG. 7), this natural product represents a promising lead for the development of new drugs against a broad range of Candida spp. infections. In contrast, keanumycin A is less active against other opportunistic pathogenic fungi such as Aspergillus fumigatus and had no effect on the growth of Rhizopus arrhizus at the concentrations tested. Interestingly, keanumycin A is extremely effective in inhibiting Botrytis cinerea (MIC=69 nM/80 μg L-1), which is a devastating phytopathogen that causes Botrytis blight, also known as gray mold, in over 1000 plant species and was ranked as the second most important plant-pathogenic fungus.61 To a lesser extent, it also inhibited the growth of Alternaria solani, the causative agent of early blight in tomatoes and potatoes, but not of Fusarium annulatum, which can infest different fruits and is additionally an opportunistic human pathogen. Inspired by the inhibition properties of keanumycin A against B. cinerea, the inventors wanted to test if the keanumycins can be used as pest control agents and protect plants from this phytopathogen. Since B. cinerea is encountered worldwide, Botrytis blight is one of the most common diseases of greenhouse crops and can cause serious economic damage. As a model organism, the inventors chose Hydrangea macrophylla, which is a flowering plant native to Asia that is used in gardening as a very popular ornamental plant and is routinely grown in greenhouses.

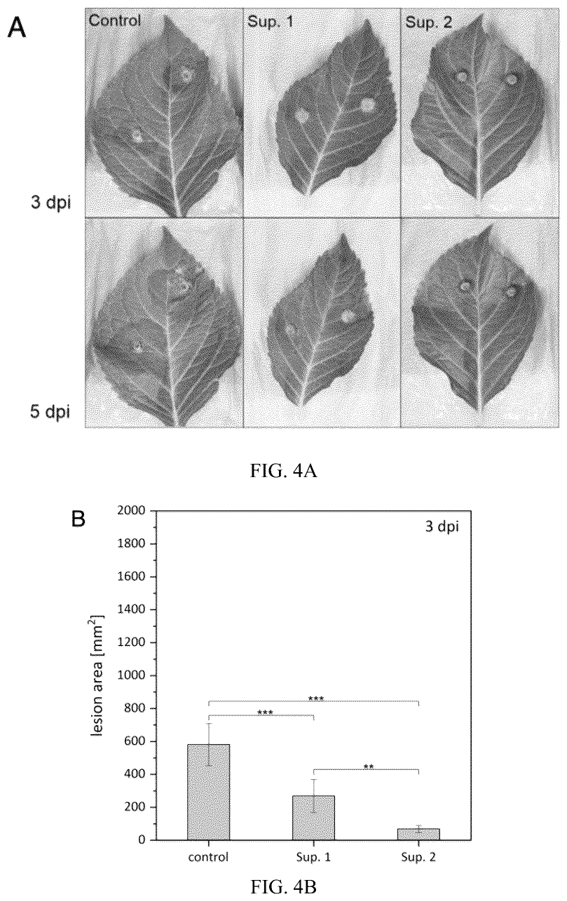

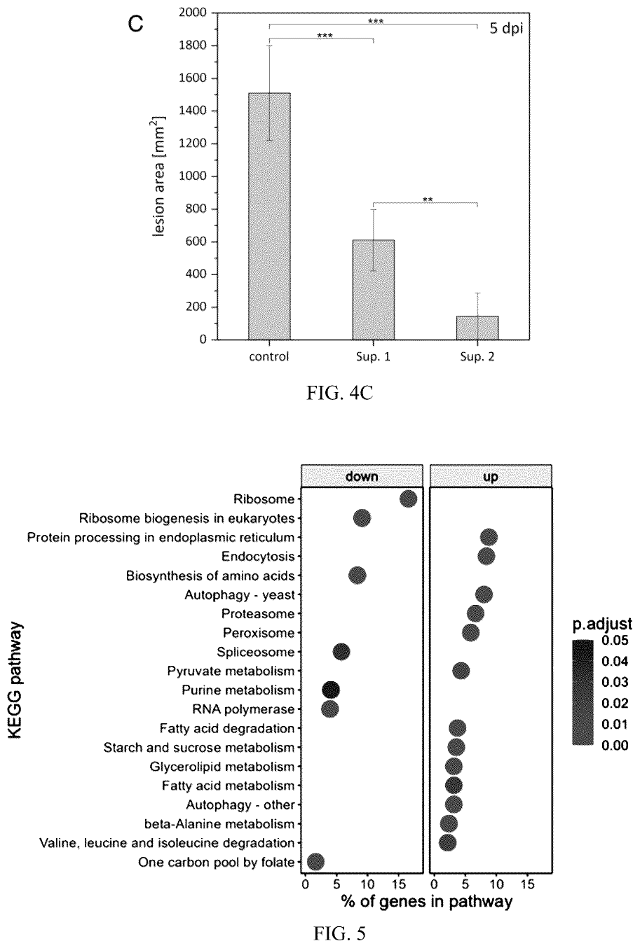

Unfortunately, isolation of keanumycin A involves multiple purification steps and only yields low amounts of pure natural product (0.1-0.2 mg L-1). Therefore, the inventors reasoned that a sterile filtrate of the fermented supernatant of a Pseudomonas sp. QS1027 mutant, which is able to biosynthesize the keanumycins but not capable of producing jessenipeptin and virginifactins, could contain sufficient amounts of highly antifungal NRLPs to inhibit the growth of B. cinerea. This assumption was supported by results of coculturing experiments on solid media. In these experiments, the wild type strain was able to inhibit growth of B. cinerea, whereas the ΔjesΔkea mutant had no effect on growth of the phytopathogen. Interestingly, the Δkea mutant, which still produces jessenipeptin, repressed the growth of the fungus albeit only slightly. In order to validate this result, the inventors determined the MIC of jessenipeptin against B. cinerea (10 mg L-1/5.2 μM), which is two orders of magnitude higher than that of keanumycin A. Indeed, when the inventors applied two different supernatants with varying concentrations of keanumycin A to mycelium plugs of B. cinerea, which were used to infect detached leaves of H. macrophylla, the inventors could see a drastic inhibition of disease-induced lesions in a concentration-dependent fashion (FIG. 4). This experiment shows that the fermentation broth of Pseudomonas sp. QS1027 can be used to fight Botrytis blight, which represents a cost-efficient, sustainable, and environmentally friendly alternative to antifungal agrochemicals. Studies on the Mechanism of Action of Keanumycin A. Mode-of-action studies of keanumycin A were facilitated by the observation that it inhibits multiple clinically relevant Candida species. Amongst those was C. albicans strain SC5314 (MIC=1.72 μM), which constitutes an important and well-studied model organism in fungal pathogenesis, allowing us to study the mode of action of keanumycin A. The inventors initially compared the transcription rate of C. albicans in the presence and absence of a sublethal keanumycin A concentration using a genome-wide microarray (FIG. 5).

General metabolic pathways were heavily impacted upon exposure to keanumycin A. Ribosome biogenesis was down-regulated, whereas protein as well as fatty acid degradation, endocytosis, and autophagy were increased, indicating a severe general stress response that showed similarities to an unfolded protein response (UPR). The UPR in C. albicans can be triggered by the amphiphilic antifungal natural product tunicamycin, which induces endoplasmic reticulum stress by inhibiting N-glycosylation causing accumulation of misfolded proteins. The protein kinase Ire1 senses those proteins, activating the UPR via the transcription factor Hac1.

To see if keanumycin A triggers the same canonical Ire1-Hac1 UPR pathway, the inventors looked at the splicing of HAC1 mRNA, which is regulated by Ire1. Compared to the positive tunicamycin control, the inventors could not observe splicing in HAC1 mRNA, which makes it unlikely that keanumycin A triggers this pathway. Since no other specific signaling pathway stood out, the inventors screened homozygous knockout mutants for significant growth differences in the absence and presence of a sublethal concentration of the NRLP. The most prominent growth differences were observed for Δmkc1, Δplb5, and Δpmt6. MKC1 codes for a crucial protein in the cell wall integrity pathway, which is a central signaling pathway for adapting to multiple cell wall stressors, indicating that keanumycin A induces cell wall stress and is able to trigger this signal transduction cascade. PLB5 is an important gene for phospholipid homeostasis and PMT6 codes for a transmembrane protein mannosyltransferase, which is involved in the construction of the outmost mannan cell wall layer in C. albicans. This demonstrated that the phospholipid composition of the fungal plasma membrane and the structural integrity of the fungal cell wall are important factors that protect fungi from the toxicity of keanumycin A. To test the inventor's assumption, the inventors focused on the altered expression of individual genes in the microarray data that influence the membrane fluidity via regulation of the biosynthesis of sphingolipids and ergosterol.

TABLE 5
Transcription Rate Change (log2-Fold Change) of Individual Genes of
C. albicans Treated with Keanumycin A Compared to Nontreated

| transcription rate change | gene | function |
|---|---|---|
| 0.66 | SYR2/SUR2 | sphingolipid biosynthesis |
| −1.10 | SCS7/FAH1 | sphingolipid biosynthesis |
| −0.44 | IPT1 | sphingolipid biosynthesis |
| 0.55 | ORM1 | sphingolipid biosynthesis |
| −1.83 | ERG3 | ergosterol biosynthesis |

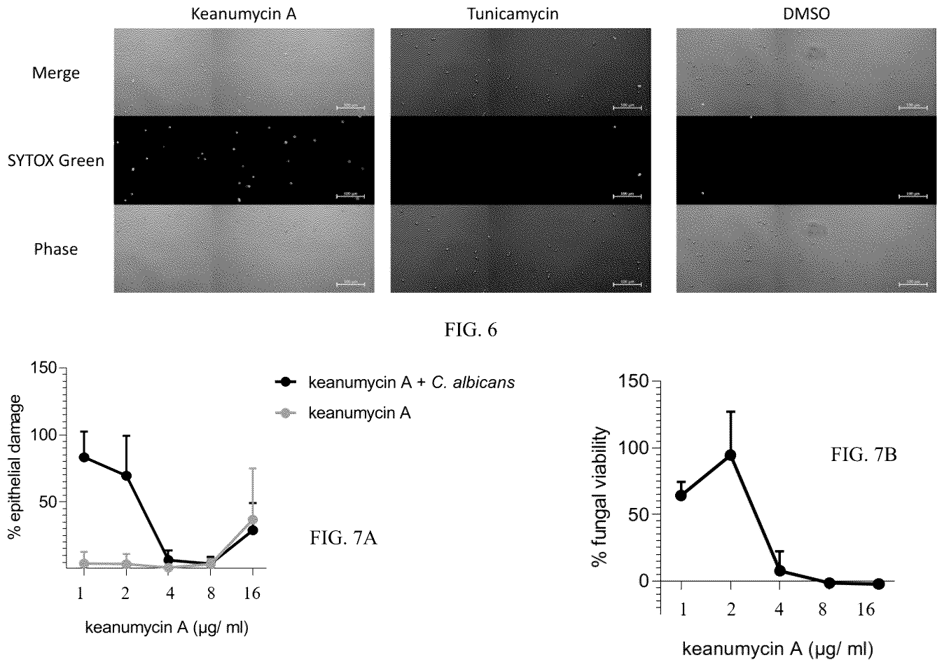

In particular, the upregulation of ORM1, which is a global repressor of the sphingolipid biosynthesis in yeasts, indicates a remodeling of membrane composition upon exposure to keanumycin A. Interestingly, the selected genes also convey resistance to syringomycin E, which belongs to the same natural product class as keanumycin A, in Saccharomyces cerevisiae. Consequently, the mechanism of action of keanumycin A is likely comparable to syringomycin E, which is an antifungal that forms ion channels in the plasma membrane leading to the collapse of the membrane potential, as it triggers a similar regulation of S. cerevisiae resistance gene homologs in C. albicans. To finally examine if keanumycin A is a membrane-active compound, the inventors performed a membrane permeabilization assay using SYTOX Green (FIG. 6). As a cell-impermeant dye, SYTOX Green enters the cell upon loss of membrane integrity and binds to DNA, which results in fluorescently stained cells. Incubation with keanumycin A led to a staining of all cells showing that this natural product increases membrane permeability. Tunicamycin on the other hand did not cause a loss of membrane integrity as the percentage of stained cells was comparable to the negative DMSO control. Thus, the keanumycins are membrane-active compounds that disturb the structure of the fungal cell wall.

### Bioactivity of Keanumycin Against Soybean Rust (SBR)

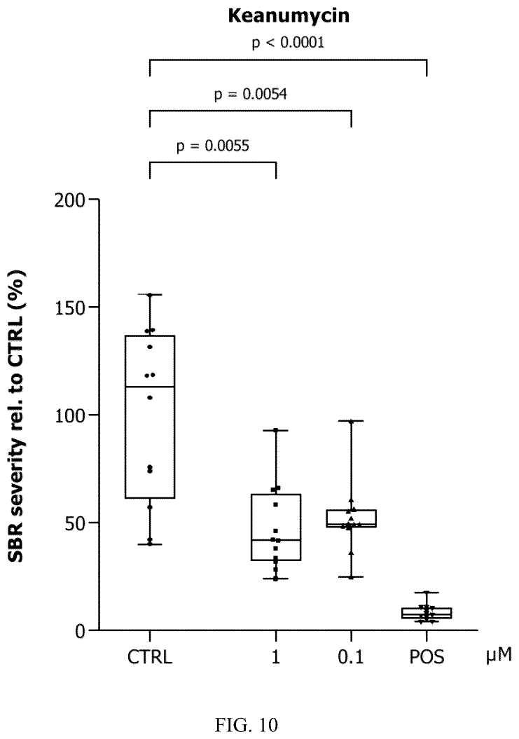

The efficiency and effect of keanumycin against soybean rust (SBR) as a preferred example for crop plants was tested on leaves of soybeans. Application of keanumycin at concentrations of 1 μM and 0.1 μM were protective, at least in part, with 24.7% (no treatment), 11.7% (1 μM), 12.9% (0.1 μM), and 2.1% (control, synthetic fungicide) of leaf areas with symptoms, respectively (see FIG. 10). Furthermore, no phototoxic effects could be identified.

1. A lipopeptide according to the following general formula 1,
(Formula 1)
X-L-Ser-D-Dab-Gly-D-Hse-L-Dab-L-allo-Thr-Dhb-L-
Asp-L-Thr
wherein
X is selected from CH₃—(CH₂)₁₄—CONH, CH₃—(CH₂)₁₂—CHOH—CH₂—CONH, and CH₃—(CH₂)₁₁—CHOH—CHOH—CH₂—CONH,
and
wherein the molecule optionally forms a cycle between the L-Ser and the last L-Thr moiety, and pharmaceutically or agrochemically acceptable salts thereof.

2. The lipopeptide according to claim 1, wherein at least one of the L-Thr is replaced by 4-Cl-L-Thr, and/or wherein the L-Asp is replaced by L-β-threo-OH-Asp.

3. The lipopeptide according to claim 1, selected from the group consisting of: 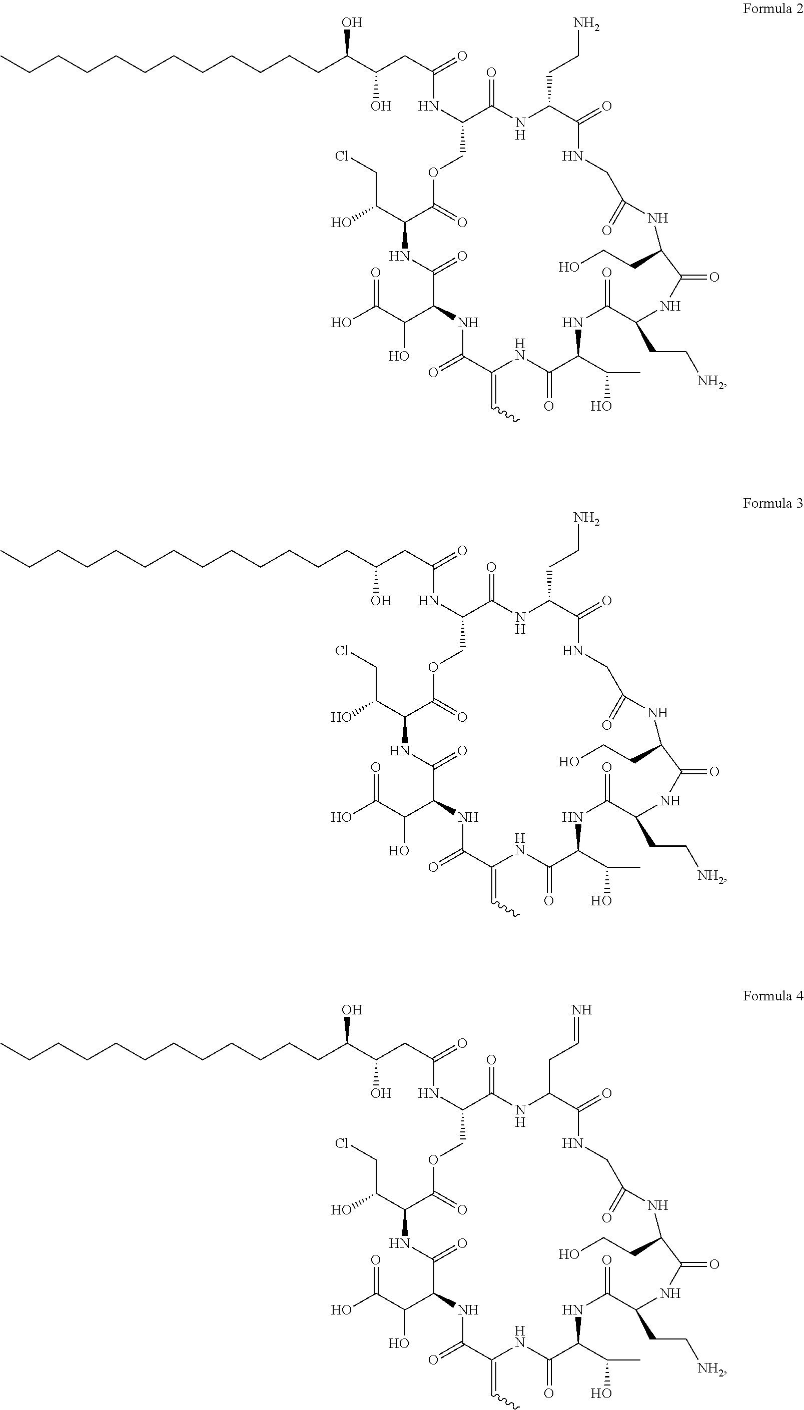
and pharmaceutically acceptable salts thereof.

4. A method for producing the lipopeptide according to claim 1, comprising:
a) culturing a strain of the bacterium Pseudomonas,
b) extracting supernatant of the culture with an organic solvent,
c) dissolving the dried extract of b) in alcohol,
d) fractioning the extract of c) using reverse phase chromatography and elution with 75% (v/v) acetonitrile in water containing 0.1% (v/v) formic acid, and
e) final purification using preparative HPLC.

5. The method for producing the lipopeptide according to claim 1, comprising chemical synthesis of the fatty acid chain and the amino acid chain comprising use of synthetic amino acid building block 2-amino-4-iminobutanoic acid.

6. A composition selected from:
A) a pharmaceutical composition or a plant protective composition comprising at least one lipopeptide according to claim 1, together with at least one pharmaceutically acceptable carrier or diluent, or at least one plant protective composition acceptable carrier or diluent and
B) an antimycotic or antiamoebal composition comprising at least one lipopeptide according to claim 1 comprising a supernatant of a culture of a strain of the bacterium Pseudomonas.

7. A method for preventing or inhibiting the growth of a fungus or an amoeba, comprising contacting the fungus or the amoeba with the composition according to claim 6.

8. The method according to claim 7, wherein said fungus is a plant pathogen and wherein said method comprises the step of applying said composition onto a plant.

9. The method according to claim 7, wherein the concentration of the lipopeptide in the composition is in a range of about 0.05 μM to about 1.5 μM.

10. A method for preventing or treating a fungal or an amoebal infection in a subject in need of said prevention or treatment, comprising administering to said subject an effective amount of the at least one lipopeptide according to claim 1.

11. The method according to claim 10, wherein the fungal infection is caused by Candida spp. Sporobolomyces salmonicolor, or Penicillium notatum or wherein the amoebal infection is caused by Acanthamoeba castellanii, or Acanthamoeba comandoni.

12. The method according to claim 4, wherein the Pseudomonas is Pseudomonas sp. QS1027, the organic solvent is n-butanol, and the alcohol is MeOH.

13. The composition according to claim 6, wherein the composition comprises a sterile filtrate of the supernatant of the Pseudomonas culture.

14. The method according to claim 8, wherein the plant pathogen is Botrytis cinerea, Phakopsora pachyrhizi or Alternaria solani.

15. The method according to claim 11, wherein the candida sp. is Candida albicans or Candida auris.
